(19) United States
(12) Patent Application Publication
(10) Pub. No.: US 2026/0365990 A1
(43) Pub. Date: Nov. 19, 2026

(54) METHOD FOR THE PRESERVATION OF FUNCTIONAL GAMETES

(71) Applicant: Julius-Maximilians-Universität Würzburg, in Vertretung des Freistaates Bayern, Würzburg (DE)

(72) Inventors: Wolfgang Schmehl, Würzburg (DE); Marcus Gutmann, Würzburg (DE); Lorenz Meinel, Würzburg (DE); Tessa Lühmann, Würzburg (DE); Simon Reiländer, Würzburg (DE); Adriane Woehl Wenigerkind, Würzburg (DE)

(73) Assignee: Julius-Maximilians-Universität Würzburg, in Vertretung des Freistaates Bayern, Würzburg (DE)

(21) Appl. No.: 19/108,125

(22) PCT Filed: Sep. 2, 2023
(86) PCT No.: PCT/EP2023/074812

§ 371 Date: Feb. 28, 2025

(30) Foreign Application Priority Data
Sep. 9, 2022 (EP) 22194868.0
Dec. 23, 2022 (EP) 22216317.2

(51) Int. Cl. C12N 5/00; A61K 33/00; A61P 15/08; C12N 5/076

(52) U.S. Cl. CPC C12N 5/526; A61K 33/00; A61P 15/08; C12N 5/061

(57) ABSTRACT

The present invention relates to the use of carbon monoxide for the preservation of gametes. The present invention provides for means and methods for the preservation of gametes. An inventive method comprises the steps of a) providing gametes or a sample comprising said gametes in a container and b) contacting said gametes in said container with carbon monoxide. The present invention also provides for the use of carbon monoxide in treating and/or preventing a disease, for example a disease caused by or linked to elevated DNA fragmentation, oxidation-reduction potential of gametes, and/or a disease of gametes caused by or linked to elevated ROS levels. Also provided is carbon monoxide for use in preventing congenital abnormality and/or aneuploidy but also the use of carbon monoxide for use in treating a disease caused by and/or linked to elevated DNA fragmentation and/or oxidation-reduction potential of gametes. The present invention furthermore relates to a method for treating congenital abnormality or aneuploidy comprising contacting carbon monoxide gas to gametes of a patient in need thereof. Thus, the inventive means, methods, and uses ensure the preservation of gametes in a functional/intact state and, thus, to safeguard a maintained and/or improved gamete quality. Such carbon monoxide treated/carbon monoxide exposed gametes are, thus, particularly useful in reproductive technologies, for example in in vitro reproductive technologies, including assisted reproductive technologies in humans and/or in (artificial) insemination techniques in animals.

The present invention relates to the use of carbon monoxide for the preservation of gametes. The present invention provides for means and methods for the preservation of gametes. An inventive method comprises the steps of a) providing gametes or a sample comprising said gametes in a container and b) contacting said gametes in said container with carbon monoxide. The present invention also provides for the use of carbon monoxide in treating and/or preventing a disease, for example a disease caused by or linked to elevated DNA fragmentation, oxidation-reduction potential of gametes, and/or a disease of gametes caused by or linked to elevated ROS levels. Also provided is carbon monoxide for use in preventing congenital abnormality and/or aneuploidy but also the use of carbon monoxide for use in treating a disease caused by and/or linked to elevated DNA fragmentation and/or oxidation-reduction potential of gametes. The present invention furthermore relates to a method for treating congenital abnormality or aneuploidy comprising contacting carbon monoxide gas to gametes of a patient in need thereof. Thus, the inventive means, methods, and uses ensure the preservation of gametes in a functional/intact state and, thus, to safeguard a maintained and/or improved gamete quality. Such carbon monoxide treated/carbon monoxide exposed gametes are, thus, particularly useful in reproductive technologies, for example in in vitro reproductive technologies, including assisted reproductive technologies in humans and/or in (artificial) insemination techniques in animals. The present invention relates to ex corpore/in vitro uses but also to in vivo uses in a medical context. In particular, desired (artificial) reproductive technologies are also described and provided herein.

Infertility is one of the most prevalent individual health problems in the world. According to estimations from the WHO about 15% of couples of reproductive age are impacted by infertility (Rutstein, Infecundity, Infertility, and Childlessness in Developing Countries. DHS Comparative Reports No. 9. Calverton, Maryland, USA. orc Macro and the World Health Organization, 2004). These cases comprise, inter alia, female, or male infertility as well as cases of infertility which comprise both, male and female infertility. About 50% of all cases are related to male infertility. Idiopathic infertility relates to about 40% of total male infertility (Mclachlan, Med J Aust 174 (2001): 116-117). 30-85% of these cases are linked to oxidative stress as a consequence of the formation of reactive oxygen species (ROS) which result in damaged/less functional male gametes/spermatozoa (e.g., by DNA fragmentation, peroxidation of lipids and apoptosis) (Mannucci, Front Mol Biosci 8 (2021): 799294). Notably, male infertility rates are increasing, and sperm quality is decreasing in healthy men, as shown by decreasing trends in sperm concentration (1.5% per year), sperm count (1.6% per year), total motility (0.4% per year), and rapid motility (5.5% per year) (Mahrukh Hameed, Infertility and Assisted Reproduction 8, 2021). Data from the International Committee for Monitoring Assisted Reproductive Technologies (ICMART) indicates a global average of 3.2 million ART cycles per year, including 1.6 million in vitro fertilizations. However, despite this high number of ART, the delivery rate stands at a low 22.5%, highlighting the need to improve access to high-quality fertility treatments for those who require it (Technologies ICfMAR, ICMART Preliminary World Report: ART, 2018). This also poses a significant burden on couples and, due to the high costs of ART procedures, also on healthcare systems worldwide. There is also a need for cost sensitive and/or accessible means and methods in reproductive technologies, in particular in ART.

The success rate of assisted reproductive technology (ART) is negatively impacted by ROS levels/damaged/non-functional gametes and results in many ART cycle failures (Zorn, Int J Androl 26 (2003): 279-285). ROS levels in an individual, for example in a human susceptible as donor for gametes to be used in ART, may increase, inter alia, through (physiological and/or psychological) stress, genetic predisposition, environmental factors (including exposition to electromagnetic waves), and/or behavioral risk factors (like, diet, smoking, alcohol consumption, and/or drug abuse; see, inter alia, Agarwal, The Lancet, 2020; Agarwal, World J Mens Health, 2020; Esteves, Andrology, 2019). Further undesired increase of ROS levels in an individual are known to the skilled artisan.

A further important health uncertainty may be a potential negative genetic alteration in newborns due to ROS-related genetic alterations in gametes, in particular sperm. For example, in natural fertilization, a DNA-damaged sperm would likely not fertilize the egg. In ART, the selection process is bypassed, which may lead to the unintended use of DNA-damaged sperm (Zini, Canadian Medical Association Journal, 175(5):495, 2006). Also in this context, ROS and resulting oxidative stress on gametes, in particular sperm/spermatozoa poses a significant burden on the success of reproductive technologies, like ART. In other words, there is a need to avoid physiological stress on gametes to be employed in such reproductive technologies. Again, one of the major corresponding physiological hurdles is the exposure of gametes in corpora as well as ex corpore to oxidative stress.

The reduction of ROS and optimization of the quality of spermatozoa has previously been proposed via the use of anti-oxidative agents which are to be administered to subjects orally. A Cochrane review from 2011 describes studies related to respective medication and concludes that the administration of these anti-oxidative agents might lead to an increased rate of birth after ART (Showell, Cochrane Database Syst Rev 1 (2011): CD007411). However, there are also disadvantages associated with the systemic administration of anti-oxidative agents such as, e.g., vitamin C, vitamin A, vitamin B complex, coenzyme Q10 and others (Sabeti, Int J Reprod Biomed 14 (2016): 231-240). First, these anti-oxidative agents need to be administered regularly and over an extended period of time (up to several months). Furthermore, these drugs can also cause side effects and are cost-intensive for patients and payers alike.

Accordingly, a technical problem underlying the present invention is the provision of functional gametes with desirable characteristics for, inter alia, reproductive technologies, like assisted reproductive technologies.

The technical problem is solved by the embodiments and items as provided herein and as specifically provided in the appended claims.

Thus, the present invention relates to means and methods for the preservation of gametes comprising the steps of
a) providing/obtaining gametes or a sample comprising said gametes in a container and
b) contacting said gametes or said sample comprising said gametes in said container with carbon monoxide.

The gametes may be contacted with/exposed to/treatment with the carbon monoxide (in particular carbon monoxide gas) for a sufficiently long time in order to preserve the gametes. Preservation of said gametes relates in particular to the preservation of gametes in a functional/intact state. Contacting the gametes with/exposing the gametes to and/or treating the gametes with carbon monoxide (in particular carbon monoxide gas) in accordance with the present invention safeguards and/or upholds, therefore, a maintained and/or an improved gamete quality. This is also explained herein below and is in particular illustrated in the appended, non-limiting examples and figures.

It is understood that the carbon monoxide (in particular the carbon monoxide gas) can be administered to the gametes directly, for examples comprised in, e.g., a buffer/or buffer system. Yet, and as also illustrated herein, the carbon monoxide (in particular the carbon monoxide gas) can also be brought into contact with said gametes comprised in a sample comprising said gametes. Said sample, may be a biological sample, like, but not limited to, in case of sperm cells/spermatozoa seminal fluid or ejaculate. It is understood that the gametes to be contacted with/exposed to/treated with carbon monoxide/carbon monoxide gas may, also be comprised in a buffer solution and/or a buffer system. The sample comprising said gametes, may also be a diluted sample. It is, e.g., envisaged that for certain applications, the seminal fluid or the ejaculate is further diluted in or diluted with a buffer or a buffer system before and during the exposure to carbon monoxide/carbon monoxide gas. Accordingly, it is understood that the term “gametes” also comprise samples, like biological samples, that comprise said gametes. The terms “contacted with”, “exposed to”, and/or “treated with” are used interchangeably in the context of the present invention.

Accordingly, the present invention relates in one embodiment to means and methods for the preservation of gametes, wherein said method may, in one embodiment, comprise the steps of
a) providing gametes in a container and
b) contacting said gametes in said container with carbon monoxide gas for a sufficiently long time to ensure preservation of gametes.

The gametes to be contacted with/exposed to/treated with carbon monoxide/carbon monoxide gas comprise sperm cells/spermatozoa or egg cells, preferably sperm cells/spermatozoa. The gametes are animal gametes, preferably but not limited to gametes from mammals, including humans. The spermatozoa to be contacted with/exposed to/treated with carbon monoxide/carbon monoxide gas may be comprised in seminal fluid, ejaculate and/or a buffer/buffer system when contacted with/exposed to/treated with carbon monoxide/carbon monoxide gas.

Carbon monoxide (CO) is an endogenous messenger molecule produced continuously in the human body. In case of exposure of the human physiologic system to stress factors, production of CO is upregulated to trigger respective defense mechanisms. Supplemented exogenously as a medicament, CO has been shown to have therapeutic potential (Motterlini, Nat Rev Drug Discov 9 (2010): 728-743), which has been proven in many preclinical studies. However, translation into regular human use is still lacking due to the lack of safe and effective systems for application of CO (Hopper, Curr Pharm Des 24 (2018): 2264-2282).

U.S. Pat. No. 9,980,981 B2 and WO 2012/096912 A1 disclose compositions comprising CO which may be used for the treatment of inflammatory diseases and neurodegenerative diseases, but not for the preservation of gametes and for their subsequent use in fertility medicine/ART. Also, WO 2022/055991 A1 discloses CO releasing formulations (“gas entrapping compositions”), which may be used for the treatment of inflammatory diseases.

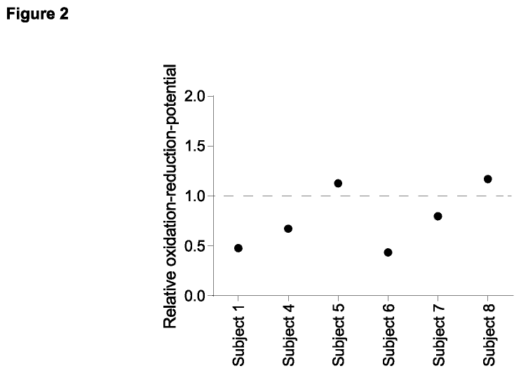

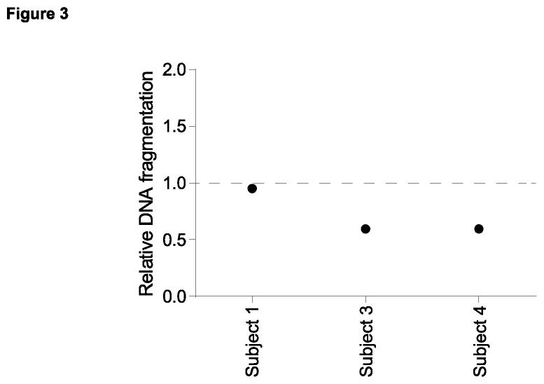

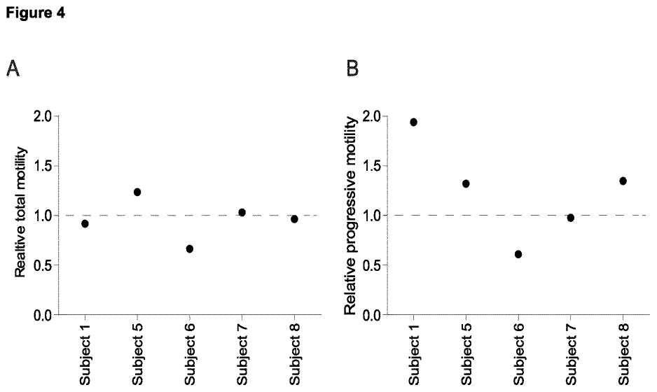

In context of the present invention, the inventors have surprisingly found that carbon monoxide (CO), in particular in form of carbon monoxide (CO) gas can successfully be used for the preservation of gametes. It was found and is illustrated and explained herein that contacting gametes with (also including exposing gametes to or treating gametes with) carbon monoxide/carbon monoxide gas ensures the preservation of said gametes, whereby said preservation means the “maintenance” in a (desired) functional/intact state. Accordingly, it was found and documented herein that carbon monoxide (CO), in particular in form of carbon monoxide (CO) gas, can safeguard/maintain and/or even improve gamete quality. For example and in particular, the inventors have found that contacting of, e.g., spermatozoa with carbon monoxide (gas) leads to (i) a reduction of ROS levels/a reduction in oxidative stress (see, e.g., appended FIGS. 2 and 5 to 9) in spermatozoa, and consequently to (ii) a reduction of DNA fragmentation (see, e.g., appended FIGS. 3, 5 to 8, and 10) in gametes, in particular spermatozoa. Furthermore, it is documented in the appended examples and figures that also (iii) an improvement of progressive motility of male gametes/spermatozoa (see, e.g., appended FIGS. 4 B and 5) compared to a control sample (not contacted with carbon monoxide) could be observed. None of these surprising technical effects have been disclosed or foreshadowed in the art. Accordingly, the inventors have found that contacting of gametes, such as spermatozoa, with carbon monoxide leads to the preservation of gametes with desirable characteristics. Desirable characteristics in context of this invention may be measurable characteristics such as, inter alia, reduced ROS levels/oxidative stress/oxidation-reduction potential, reduced levels of DNA fragmentation, improved progressive motility, and/or desired maintenance of motility, inter alia, in form of average path velocity (VAP) of spermatozoa. The corresponding improvements are documented and illustrated in the appended examples and are in particular documented in comparison to a control sample of gametes that was not incubated with/brought into contact with CO. The preserved gametes exhibiting one or more of the above-described desirable characteristics may subsequently, for example, be used in assisted reproductive technology applications. Therefore, the means, methods, and uses of the present invention also obviate the need for continued systemic administration of (potentially harmful) anti-oxidative agents to subjects/patients participating in such applications. Moreover, due to their gamete-preserving effect, the means, methods, and uses of the present invention may also be employed to preserve/store gametes during prolonged (gamete) handling-times during ART applications. The herein provided means, methods, and uses are not only relevant in ex corpore or in vitro uses but also in in vivo methods and in vivo uses of carbon monoxide. As for example described herein, carbon monoxide can also be used to improve gamete quality in vivo. It is, inter alia, envisaged that male individuals are treated with CO in order to avoid for example DNA fragmentation on gametes due to physiologically damaging or potentially damaging oxidative stress. This can, inter alia, be achieved by administering to the individual in need of such treatment the carbon monoxide via patches that release CO. This embodiment of the invention is further illustrated herein below and relates, inter alia, to the inventive use of carbon monoxide in treating and/or preventing a disease that is causative for undesired (genetic) disorders of the developing embryo and/or fetus. For example, the present invention provides also for the medical use of carbon monoxide for use in preventing congenital abnormality and/or aneuploidy. The present invention further provides, for example, also for the medical use of carbon monoxide in treating and/or reducing male infertility. For example, the present invention provides also for the medical use of carbon monoxide in treating and/or preventing a disease caused by/linked to elevated DNA fragmentation and/or oxidation-reduction potential of gametes. Further, the present invention provides, for example, also for the medical use of carbon monoxide in treating and/or preventing a disease of gametes caused by/linked to elevated ROS levels. Also, this embodiment is further discussed herein below. The gist of the invention is the avoidance of ROS mediated physiological damage.

The present invention in its broadest embodiment relates to the preservation of gametes using carbon monoxide. The gametes may be contacted with/exposed to/treated with said carbon monoxide in vitro, ex vivo and/or ex corpore. However, as explained below, also in vivo uses, for example for medical intervention, are described and disclosed herein and are part of the present invention.

The term “preservation” within the means, methods, and uses of the present invention refers to a method, activity, or process of keeping gametes functional, alive, intact, and/or free from damage and/or decay using carbon monoxide/carbon monoxide gas. This in particular refers to the time after the gametes have been collected from a subject, whereby the subject may be a healthy or a diseased subject. The subject is preferably a mammal, most preferably a human. Yet, the means and methods, as well as the uses, of the present invention are also readily applicable to gametes, in particular spermatozoa, of other animals, like birds, fish and reptiles. The means and methods of the present invention, i.e., contacting gametes with carbon monoxide, can ensure the preservation of gametes in a functional/intact state. One of the gists of the present invention is also illustrated in the appended examples wherein it is shown that carbon monoxide exposure to gametes, in particular to spermatozoa, upholds a maintained and/or improved gamete quality. The preserved gametes may successfully be used in, e.g. reproductive medicine, in particular in assisted reproductive technology (ART) applications but also in technologies like artificial inseminations of, e.g., farm animals. Accordingly, the terms “gamete preservation” and “preservation of gamete quality” are to be understood to refer to keeping gametes functional, alive, intact, and/or free from damage and/or decay for a given time as compared to a reference timepoint. The reference timepoint may be defined by the timepoint at which the gametes are collected from a (healthy or diseased) subject, such as a male or a female human subject for example. The term “preservation” may however also be understood in the context of temporary ex vivo storage of gametes during prolonged (gamete) handling times. This may in particular be the case in the context of ART applications. The term “ex vivo” is known to the person skilled in the art, and refer especially, also in the context of the present invention, to exposure of the gametes to be reserved in context with this invention to carbon monoxide (CO) in an ex corpore setting. In other words, the gametes in particular the spermatozoa are exposed to the carbon monoxide outside of the individuals' body, for example after ejaculation into a corresponding (collection) container. The individual is, therefore, preferably an individual capable of producing corresponding gametes/spermatozoa, i.e., sperm. Ejaculation into corresponding containers are for example relevant in (artificial) reproduction technologies, like in vitro fertilization technologies. It is understood that the direct ejaculation into a container is not a prerequisite of the means, methods, and uses of the present invention. It is also envisaged that the present invention is employed onto any seminal fluid, semen, ejaculate, sperm sample, isolated and further purified sperm, or spermatozoa and also on corresponding diluted and/or further treated, processed, handled, liquified, previously frozen, thawing or thawed biological samples comprising the gametes, preferably the spermatozoa to be treated. The present invention is also useful and be readily applied during the handling of any biological sample comprising the gametes, preferably the spermatozoa to be preserved in context of this invention. Accordingly, it is also envisaged that any biological sample comprising the gametes are exposed to carbon monoxide/carbon monoxide gas during any manipulation or handling of said biological sample. In the context of the present invention, the term “ex vivo” may be used interchangeably with “ex corpore”. Corresponding ex vivo and ex corpore techniques and methods can be considered as “in vitro” techniques/methods. Accordingly, in one embodiment, an ex vivo treatment may refer to the treatment of gametes that are provided in a container with carbon monoxide or the treatment of gametes that are already in a container and that are then exposed to the carbon monoxide. However, in another embodiment of the invention described herein, the inventive concept of exposing gametes to carbon monoxide in order to beneficially preserve said gametes may also be employed to the human or animal body. Accordingly, the present invention also relates to the medical use of carbon monoxide in methods of treatment or methods of prevention of disease whereby these methods comprise the exposure of gametes within the human or animal body. These medical uses also comprise, accordingly, in vivo uses of the carbon monoxide. The corresponding exposure of carbon monoxide to gametes may, for example and non-limiting, be obtained by (medical) patches, as also described herein below; see also the provision of corresponding medical patches releasing carbon monoxide as described in WO 2021/180908 A1 and Ruopp et al. (2023, Journal of Controlled Release). In one particular aspect, an in vivo treatment/exposure also refers to the exposure of gametes (in particular spermatozoa) to carbon monoxide, whereby said gametes are still present in the gonads of an organism, preferably in an eukaryote, more preferably in a (male) mammal, most preferably in a human (man).

The terms “functional, alive, intact, and/or free from damage and/or decay” in context of the present invention relate to one or more characteristics of the gametes which may be negatively influenced by, for example, reactive oxygen species (ROS). These characteristics may be preserved by the means, methods, and uses of the present invention and may, inter alia, comprise “desired/desirable characteristics” as described herein above. For example, as is also illustrated in appended Examples 2 and 3 and FIGS. 2 to 7, non-limiting (desired) characteristics of the gametes may comprise (reduced) oxidation-reduction potential/ROS levels, (reduced) DNA-fragmentation, (increased) motility (in case of sperms/spermatozoa), in particular (increased) progressive motility, (reduced) peroxidation of lipids, (reduced) apoptosis, and (reduced) degeneration of sperm. A person skilled in the art is aware of means and methods to determine such (desirable) characteristics from gametes. Exemplary methods and commercial kits to determine in particular (static) oxidation-reduction potential/ROS levels, DNA-fragmentation and total and progressive motility of spermatozoa are however also described in detail in Examples 2 and 3. Example 3 further describes measurements of average path velocity (VAP) of spermatozoa. VAP is defined as the speed sperms are moving, measured in microns per second (μm/s) and, together with total motility and progressive motility, is an important parameter to describe overall motility of spermatozoa. According to the present invention, a further desired characteristics of gametes may be (maintained/increased) VAP.

For example, the Male Infertility Oxidative System (MiOXSYS, Englewood, CO) may be used to determine (static) oxidation-reduction potential/ROS levels of spermatozoa, the Halo Sperm G2 Kit (Halotech, Madrid, Spain) may be used to determine DNA-fragmentation of spermatozoa and the CEROS II Computer Assisted Sperm Analysis (CASA) system (Hamilton Thorne, Beverly, MA) may be used to determine sperm motility, in particular total and progressive motility and VAP.

Thus, (CO—) preserved (functional, alive, or intact) gametes in accordance with the present invention may be gametes that exhibit desirable characteristics. For example, gametes contacted with CO may, inter alia, exhibit (i) less oxidation-reduction-potential, (ii) less DNA fragmentation/DNA damage and (iii) a higher or unaltered/maintained progressive motility compared to unpreserved gametes (i.e., gametes that were not contacted with CO). Graphical representations of such (desirable) characteristics are, for example, also illustrated in appended FIGS. 2 to 10. CO-preserved gametes may be gametes comprising a haploid set of chromosomes with significantly less or without chromosomal abnormalities as compared to unpreserved gametes. Chromosomal abnormalities may, inter alia, be selected from the group consisting of numerical abnormality (e.g., aneuploidy), structural abnormalities (e.g., deletions, duplications, inversions, insertions, translocations, rings, isochromosomes) and/or acquired chromosome abnormalities. In accordance with the present invention, gametes may be healthy (no chromosomal abnormalities) or diseased gametes (chromosomal abnormalities).

In context of the present invention, the above term “successfully be used” refers to the use of said preserved gametes in reproductive medicine, in particular in ART applications or in (artificial) insemination processes. The use of these preserved gametes as obtained by the means, methods, and uses of the present invention in these application(s) is specifically associated with a higher likelihood of a positive (i.e., a desired) outcome of said application(s). For example, CO-preserved functional/alive/intact haploid gametes (in contrast to unpreserved gametes) may lead to the formation of a healthy diploid zygote with increased likelihood (corresponding to the desired outcome) during in vitro fertilization (IVF), which can give rise to normal blastocyst formation and the establishment of normal pregnancy (i.e., a pregnancy without complications that results in the birth of a healthy newborn whose risk to suffer from congenital abnormality or aneuploidy is significantly reduced).

A “zygote” in context of this invention is a eukaryotic cell formed by a fertilization event between two gametes. The zygote's genome is a combination of the DNA in each gamete and contains all of the genetic information of a new individual organism. In multicellular organisms, the zygote is the earliest developmental stage.

During human fertilization, a released ovum (a haploid secondary oocyte/egg cell with replicate chromosome copies; female gamete) and a haploid sperm cell (male gamete) combine to form a single diploid cell called the zygote. Once the single sperm fuses with the egg cell, the latter completes the division of the second meiosis forming a haploid daughter with only 23 chromosomes, almost all of the cytoplasm, and the male pronucleus. The other product of meiosis is the second polar body with only chromosomes but no ability to replicate or survive. In the fertilized daughter cell, DNA is then replicated in the two separate pronuclei derived from the sperm and ovum, making the zygote's chromosome number temporarily 4n diploid. After approximately 30 hours from the time of fertilization, a fusion of the pronuclei and immediate mitotic division produce two diploid daughter cells called blastomeres which give rise to a blastocyst which finally forms an embryo.

The term “healthy” above refers to the diploid zygote and is to be understood in contrast to a diseased/abnormal zygote.

A diseased/abnormal zygote may, for example, contain damaged/fragmented DNA or may not be diploid as a consequence of the fusion of a sperm and an egg cell of which at least one may be (an) euploid. A diseased/abnormal zygote may either not give rise to blastomere/blastocyst formation and the formation of an embryo or it may give rise to blastomere/blastocyst formation and the establishment of an embryo, but it may lead to miscarriage or the birth of a newborn who suffers from congenital abnormality or aneuploidy (or euploidy) at a higher probability as compared to a healthy zygote. Non-limiting examples of congenital abnormality or aneuploidy are described herein further down below.

Hence, a “healthy” zygote is diploid (i.e., comprises a complete set of paternal and maternal chromosomes) and can lead to normal blastomere/blastocyst formation/formation of an embryo. A healthy zygote may thus lead to the birth of a newborn without congenital abnormality or aneuploidy (or euploidy) at a higher probability as compared to a diseased/abnormal zygote.

For example, in accordance with the means, methods, and uses of the present invention, a gamete may be a healthy (does not comprise one or more chromosomal abnormalities) or a diseased gamete (does comprise one or more chromosomal abnormalities). A gamete defines a haploid cell that fuses with another haploid cell during fertilization in organisms that reproduce sexually. Gametes are an organism's reproductive cells, also commonly referred to as sex cells. In species that produce two morphologically distinct types of gametes (as is, for example, the case in mammals, such as humans), and in which each individual/sex produces only one type of gametes, a female is any individual that produces the larger type of gamete (called an oocyte/egg cell) and a male produces the smaller type (called a sperm cell/spermatozoon). Thus, in context of the means, methods, and uses of the present invention, gametes comprise spermatozoa/sperm cells and/or egg cells/oocytes, preferably spermatozoa/sperm cells and/or egg cells/oocytes which have been collected ex-vivo from a mammalian subject like a male or a female human subject. Said mammalian subject, like a male or a female human subject, may be healthy or may suffer from/be predisposed to disease like, for example, infertility or chromosomal abnormalities. In context of the present invention, it may be preferred that spermatozoa may be comprised within seminal fluid, ejaculate and/or a buffer/buffer system which may allow the survival of the sperm cells. Accordingly, the gametes to be contacted with carbon monoxide can also be comprised in a biological sample or in a buffer/buffer system.

The skilled artisan is aware of exemplary buffers/buffer systems suitable for, inter alia but not limiting to, diluting, storing, washing, handling, treating, buffering, purifying gametes, in particular spermatozoa. Non-limiting examples of such buffers/buffer systems include, e.g., sperm dilution buffers, sperm maintenance buffers, and sperm washing buffers. Such buffers/buffer systems may comprise, inter alia, water/watery solutions, salts/ions, buffer reagents/buffer solutions, amino acids (inter alia, glycine and/or taurine), energy substrates, and antibiotics (inter alia, gentamycin sulfate). Non-limiting examples of buffer reagents/buffer solutions may include HEPES (4-(2-hydroxyethyl)-1-piperazineethanesulfonic acid), Sodium Bicarbonate, MOPS (3-(N-morpholino) propanesulfonic acid), and phosphate-buffered saline (PBS). Non-limiting examples of salts/ions may include potassium phosphate, sodium chloride, potassium chloride, calcium chloride, and magnesium sulfate. Non-limiting examples of energy substrates may include sodium lactate, sodium pyruvate, and glucose. The person skilled in the art of artificial reproduction technologies or a related field of work, is aware to design/generate suitable buffers/buffer systems comprising, inter alia, the herein above described exemplary components/constituents. Alternatively, comparable/functionally equivalent buffers/buffer systems, such as, e.g., Multipurpose Handling Medium Complete (FUJIFILM) and PureSperm®Wash (Nidacon) are readily commercially available to the skilled artisan.

Spermatozoa/sperm cells and egg cells/oocytes are the male and female reproductive cells (“gametes”) in anisogamous forms of mammalian sexual reproduction, respectively.

Sperm cells form during a process known as spermatogenesis, which in mammals takes place in the seminiferous tubules of the testes. This process involves the production of several successive sperm cell precursors, starting with spermatogonia, which differentiate into spermatocytes. The spermatocytes then undergo meiosis, reducing their chromosome number by half, which produces spermatids. The spermatids then mature and, in mammals, form a tail, or flagellum, which gives rise to the mature, motile sperm cell.

Their movement may either be described by the term “progressive motility” which refers to sperm cells/spermatozoa that are moving/swimming in a mostly straight line or large circles. The term “non-progressive motility” on the other hand refers to sperm cells/spermatozoa that do not move/swim in straight lines or that swim in very tight circles. Both, progressive motility, and non-progressive motility describe the total motility of a sperm cell. Each of these motilities (progressive, non-progressive and total motility) can be measured using standard techniques which are known in the art. In the appended Examples 2 and 3, the CEROS II (Hamilton Thorne, Beverly, MA) computer assisted sperm analysis (CASA) was, for example, used to determine total and progressive motility of sperm cells/spermatozoa.

Sperm cells cannot divide and have a limited lifespan, but after fusion with egg cells during fertilization, a new organism/embryo begins to develop, starting as a totipotent zygote. The human sperm cell is haploid (n), so that its 23 chromosomes can join the 23 chromosomes of the female egg cell to form a diploid (2n) cell with 46 paired chromosomes. In mammals, sperm cells/spermatozoa are stored in the epididymis and are released from the penis during ejaculation in a fluid known as seminal fluid/semen/ejaculate.

Sperm cell quantity and quality are important measures of male fertility. However, the genetic quality of sperm cells, as well as their motility, typically decrease with age. DNA damages present in, for example, sperm cells in the period after meiosis but before fertilization (i.e., the period that the present invention is primarily focusing on) can have serious deleterious effects on fertility and on the developing embryo. Mammalian sperm cells, in particular also human sperm cells, are particularly vulnerable to free radical attack (by e.g., reactive oxygen species (ROS)) and the resulting oxidative damage. Basis for this vulnerability is the progressive loss of DNA damage repair mechanisms during spermatogenesis. Therefore, the means, methods, and uses of the present invention, i.e., the contacting of gametes like spermatozoa/sperm cells with carbon monoxide gas, prevent such DNA damages (in particular in the period after meiosis but before fertilization). This is also evident, for example, from appended FIGS. 3, and 5 to 10 which clearly indicate that the degree of DNA damage/DNA fragmentation is higher in sperm samples that were not contacted with carbon monoxide as compared to sperm samples obtained from the same subject which were contacted with carbon monoxide.

Egg cells/oocytes are produced in the ovary during female gametogenesis, also termed oogenesis. During oogenesis, secondary oocytes are generated which are haploid and in which meiosis II may be halted at the metaphase II stage until fertilization. Thus, in accordance with the present invention, egg cells/oocytes may be secondary oocytes.

Both, spermatozoa/sperm cells and egg cells/oocytes may be collected from a mammalian subject such as a healthy or diseased human subject prior to being contacted with carbon monoxide gas. Spermatozoa/sperm cells/semen may, for example, be obtained from the epididymis by ejaculation into a (sterile) container during masturbation, condom collection or epididymal extraction. Egg cells/oocytes on the other hand may, for example, be obtained by transvaginal oocyte retrieval.

Accordingly, in a preferred embodiment of the present invention, gametes may be healthy or diseased human gametes collected/obtained from a healthy or diseased male or a female subject. In a different aspect, gametes may however also be collected/obtained from other mammals, in particular also from, inter alia, cattle, horses, pigs, sheep, goats, camels, alpacas, dogs, cats and the like. In another aspect, gametes may also be collected/obtained from non-mammalian animals, in particular also from, inter alia, birds, fish, reptiles, and the like, including but not limited to falcons and/or carps.

After collection, collected gametes are to be provided in a container in accordance with the means, methods, and uses of the present invention. Such a container may be a container that is open or a container that is preferably lockable/sealable (airtight). In one specific aspect, such a container may have a valve for connecting a CO-transporting tube to the container. A container to be used in accordance with the means, methods, and uses of the present invention may be a container constructed from (a) polymer(s), elastomer(s), metal(s), glass(es) or ceramic(s) but polymer(s) are/is generally preferred. In one aspect the container may be constructed from polypropylene (PP), polyethylene (PE) or polystyrole (PS). In one aspect a container to be used may be sterile/sterilized, i.e., the container may be clean and free from germs. Exemplary containers that have also been used in the appended examples may be sample vials or gas-tight Falcon tubes. Another container to be used may be a (sterile) specimen cup. The person skilled in the art is fully aware of containers to be used in the context of this invention.

In one embodiment, seminal fluid/semen/ejaculate may be liquified. Liquefaction may be carried out before and/or while gametes, in particular spermatozoa as comprised in seminal fluid/semen/ejaculate are contacted with carbon monoxide gas for preservation. Liquefaction in this context describes the process of braking up the gel formed by proteins from the seminal vesicles and the prostate in order for the seminal fluid/semen/ejaculate to become more liquid. A person skilled in the art is aware of means and methods to perform liquefaction. For example, and routinely, liquefaction takes place naturally during seminal fluid/semen/ejaculate incubation at 25 °C for 30 to 60 minutes as is also evident from appended Example 2.

In accordance with the means, methods, and uses of the present invention, the gametes provided in a container are to be contacted with carbon monoxide gas for a sufficiently long time to ensure preservation of gametes.

In a preferred embodiment, gametes are contacted with carbon monoxide, in particular in form of CO gas. In such a case, gametes are first provided in a container and CO gas or a CO-releasing system releasing CO gas (activated CORS) is added to the container comprising the gametes in a second step, like step b) above. The present invention further relates to means, methods, and uses that allow for contacting said gametes with carbon monoxide less than 15, 14, 13, 12, 11, 10, 9, 8, 7, 6, 5, 4, 3, 2, or 1 min after collection of said gametes in a container. However, it may also be envisioned that CO gas is directly contacted with gametes and/or with (biological) samples comprising the same. Said samples may comprise, in all embodiments of this invention, seminal fluid, ejaculate and/or a buffer or a buffer system comprising the gametes, in particular the spermatozoa. Carbon monoxide, in particular CO gas may be first provided in a container and the gametes may be added to the container comprising the carbon monoxide/CO gas in a second step. As such, the gametes may be contacted with carbon monoxide immediately after collection of the gametes (or after collection and/or provision of a sample comprising the same, like seminal fluid, ejaculate and/or a buffer or a buffer system comprising the gametes) in a container. It is evident, inter alia, from the examples that providing carbon monoxide in a container may comprise activating a carbon monoxide releasing system (CORS) prior to the collection of gametes in a container, resulting in carbon monoxide formation in said container. It is evident that such a container may be sealed/closed between CORS activation and the collection of said gametes. In the context of the present invention, an exemplary carbon monoxide releasing system may be activated about 10, about 9, about 8, about 7, about 6, about 5, about 4, about 3, about 2, or about 1 min before the collection of said gametes. In other words, the carbon monoxide/CO gas may be provided in a first step in or to a container and the gametes or the sample comprising said gametes may be added in a second step. Vice versa, it is also within the scope of the present invention that the gametes (or the sample comprising said gametes) are first provided in a container and in a second step the carbon monoxide/CO gas is added to said container. The addition of the carbon monoxide may also involve a carbon monoxide releasing system (i.e., a CORS).

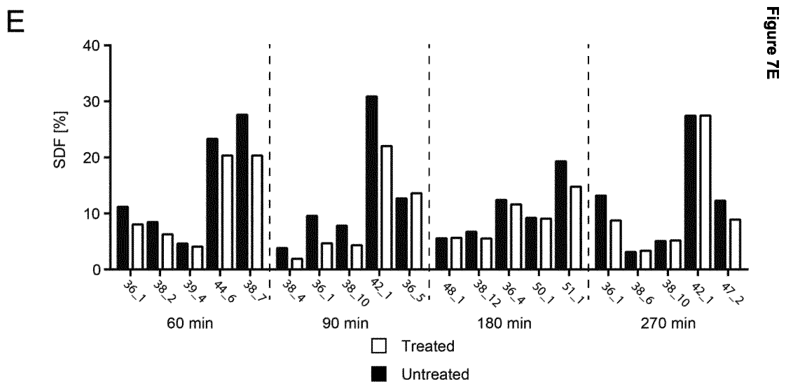

It is also envisaged that the gametes/or a sample comprising the gametes are brought into contact with carbon monoxide in a repetitive or repeated manner. In other words, the gametes/or a sample comprising the gametes may be contacted with carbon monoxide multiple times. For example (and non-limiting), it is also envisaged that the gametes are brought into contact with carbon monoxide/CO gas after at least one freezing-thawing cycle. Therefore, it is also within the context of this invention that gametes are contacted with carbon monoxide/CO gas after said gametes or said sample comprising the same, had been conserved (for example by freezing). The exposure of the gametes or the samples comprising the gametes, may, inter alia, occur during and/or after the conservation is lifted, for example by thawing of the frozen gametes/the frozen sample comprising the gametes. Corresponding freezing and or thawing protocols are very well known in the art (for example in but not limited to, reproductive methods, like artificial reproductive technologies, in vitro fertilizations, artificial inseminations, etc.). Such protocols can be combined with the teachings of the present invention, i.e., the gametes or samples comprising the same can be exposed to carbon monoxide during and after such protocol steps in order to preserve the gametes as described and illustrated herein. Accordingly, the present invention also relates to means, methods and uses in which frozen and/or thawed gametes, in particular spermatozoa, or frozen and/or thawed biological samples, like seminal fluids, ejaculates or buffers/buffer systems comprising said gametes are brought into contact with carbon monoxide. In other words, the present invention is not only applicable on “fresh” gametes or (biological) samples or buffers/buffer systems comprising the same, but also on gametes and biological samples/buffers/buffer systems comprising the same which had been conserved, like by freezing. The carbon monoxide is preferably used in a sufficient time/a sufficiently long time to enable and/or ensure preservation of gametes. This sufficient time/sufficiently long time may be or may comprise the time or the time period starting from collecting or obtaining the gametes or the sample that comprises the gametes, preferably the spermatozoa, to be preserved until their further use, for example in artificial reproductive technologies, in in vitro fertilizations, (artificial) inseminations of e.g., farm animals. Accordingly, said “sufficient time” relates to the time period in which said gametes are or the sample comprising said gametes is exposed to the carbon monoxide/carbon monoxide gas. The (ensured) preservation of said gametes also relates to the maintenance of a positive and/or healthy physiological status, for example but not limited to, a low oxidative burden (low ROS status). The (ensured) preservation also relates to the further avoidance of additional and/or undesired effects on the gametes, like exposure to further inherent or external adverse events, like oxidative stress (e.g., ROS). Such undesired effects and adverse events can have a negative effect on the gametes, in particular on spermatozoa, and can negatively influence the “quality” of these gametes. Accordingly, and as illustrated in the appended examples, the herein described “preservation of gametes”, also relates a “preservation” of a desired quality of said gametes. This quality is, inter alia, reflected by desired motility of the gametes (i.e., spermatozoa), by a low or even nonexistent DNA-fragmentation/DNA-fragmentation rate, by a medically, clinically, or biologically unremarkable spermiogram, or by a low oxidative stress level. Accordingly, the “preservation” and/or the “preservation status” of these gametes, may, inter alia but not limited to, be assessed via determination of motility and/or motility capacity of said gametes (in particular of sperm cells/spermatozoa), DNA-fragmentation status of said gametes, by spermiogram(s) and/or determination of the oxidation-reduction potential of said gametes. Corresponding methods are well known in the art and readily available, to the skilled artisan, like individuals working in reproductive medicine/ART, but also by individuals employing (artificial) inseminations/insemination protocols, for example on farm animals. Corresponding assessment methods for the determination of the “quality” of the gametes, like the preservation of the gametes in a desired status, are also illustrated in appended examples. It is documented in these examples that the present invention ensures a preservation of the gametes, in particular of spermatozoa. Said preservation may comprise the maintenance of a (desired) healthy status of the gametes but, as also documented herein, said preservation via carbon monoxide may also provide for an improvement in the quality of the gametes. It was, inter alia, surprisingly be shown that the exposure of gametes, in particular of sperm cells/spermatozoa to carbon monoxide may even augment/ameliorate the quality of said gametes, in particular of sperm cells/spermatozoa; see, e.g., appended examples 2 and 3 in which “sperm/spermatozoa quality” in individual subjects is comparatively assessed of samples treated with/exposed to carbon monoxide versus samples of the same subject/individual/donor that were not exposed to/treated with carbon monoxide. Corresponding, illustrative results are also shown, inter alia, in appended FIG. 3 (see in particular “subjects” 3 and 4, documenting improved quality, i.e., less DNA fragmentation between samples of the same individuals treated with carbon monoxide versus the sample(s) not treated/exposed to carbon monoxide. The gamete sample of the same donor was separated in two equal aliquots and the only difference was exposure to carbon monoxide or no exposure to carbon monoxide. Also appended FIG. 7 D shows a surprising improvement of the gamete quality (here sperm cell/spermatozoa quality). In FIG. 7 D it is illustrated that negative influences of inherent (but also of external) oxidative stress can be avoided by the means and methods of the present invention, i.e., by exposure of the gametes/samples comprising the same to carbon monoxide. It could surprisingly be shown that a reduced oxidation-reduction potential is observed in the carbon monoxide exposed/carbon monoxide treated gamete samples/aliquots as compared to the non-treated samples/aliquots comprising the sperm cells/spermatozoa of the same subject/individual/donor; see, e.g., and in particular, Subjects 42_1 or 38_7. However, a similar effect could also be observed in other subjects.

It is evident that the herein illustrated maintenance and/or even a certain, but relevant improvement of the quality of the gametes is brought about by the contacting of/exposure of/treatment of gametes, in particular spermatozoa to carbon monoxide. Accordingly, the present invention, in general, relates to the preservation of gametes in a desired status, for example in a quality that is acceptable for use of the gametes in artificial reproductive technology and the like. Therefore, said preservation of the gametes may comprise the maintenance of a given quality of the gametes but may also comprise an improvement of the gamete quality. Said preservation is in particular of importance in the herein described in vitro/ex corpore artificial reproductive technology methods, e.g., in the preparation and ex corpore handling of the gametes for such artificial reproductive technology methods and/or in (artificial) insemination protocols. Accordingly, “preservation”/“maintenance” of the gametes means in particular, but non-limiting, the preservation/maintenance of the gametes' quality during ex corpore handling and/or in in vitro settings. It is, inter alia but not limiting, illustrated herein that the preservation in the sense of the present invention can successfully by employed in in vitro/ex corpore applications wherein the handling of gametes is necessary. An example is the provision of sperm/spermatozoa, of (biological) samples, or of buffers/buffer systems comprising the same for artificial reproductive technology, like in vitro fertilizations. It could be documented herein in the appended examples that the tested and carbon monoxide treated spermatozoa maintained (or even improved) their quality over a relatively long ex corpore handling time. It has been shown that even up to 1.5 or even 2 hours after sample collection and corresponding exposure to carbon monoxide the gamete quality was satisfactory or even improved (as compared to non-treated samples). Accordingly, and in a non-limiting embodiment, it is envisaged that the gametes (or the samples/buffers/buffer systems comprising the same) are contacted with said carbon monoxide for about 15 to about 120 minutes, preferably for about 30 to about 90 minutes, more preferably for about 30 to about 60 minutes. This contacting of/exposure to/treatment of the gametes (or the samples/buffers/buffer systems comprising the same) with carbon monoxide may occur at room temperature, i.e., at about 25° to about 35 °C. The maintenance of a given gamete quality or, to a certain extent, even the improvement of the quality of the gametes in certain ex corpore/in vitro situations is one of the (non-limiting) advantages of the present invention. It is documented that even during a certain, even relatively long (in vitro) handling time (up to even 2 hours) at room temperature the preservation of the gametes is ensured, i.e., their quality is maintained. Accordingly, means and methods are provided herein for the preservation of gametes wherein said gametes (or samples/buffers/buffer systems comprising said gametes) are contacted with carbon monoxide/carbon monoxide gas in order to ensure preservation of said gametes/in order to maintain the quality of said gametes. Said means and methods may comprise contacting/treating/exposing the gametes (or the sample/buffers/buffer systems comprising the same) with the carbon monoxide/carbon monoxide gas for about 15 to about 120 minutes, preferably for about 30 to about 90 minutes, more preferably for about 30 to about 60 minutes. Said contacting/treatment with/exposure to carbon monoxide/carbon monoxide gas may occur at about 25 to about 35 °C.

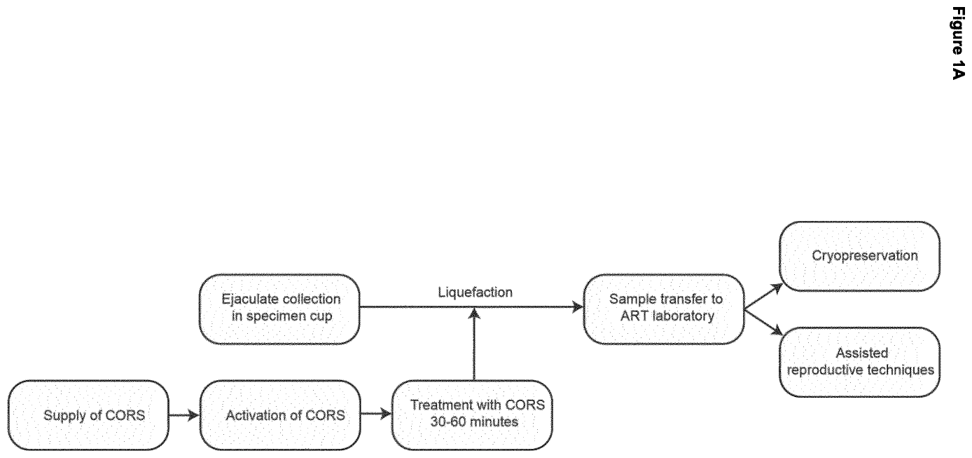

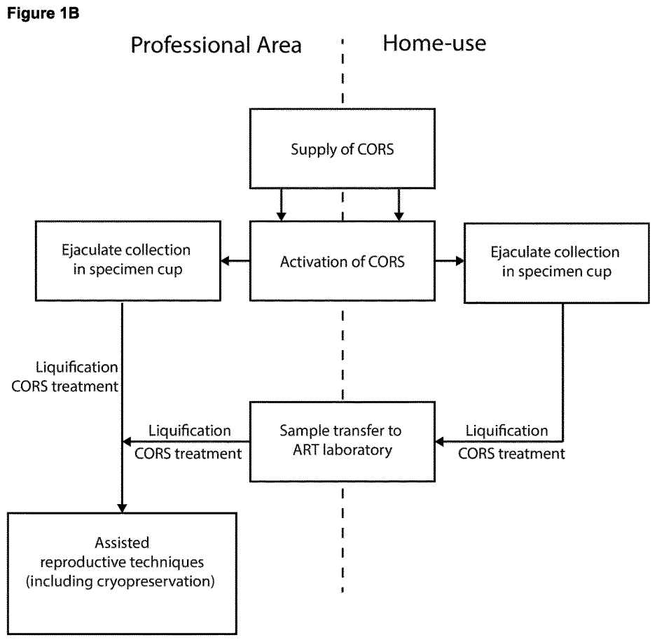

The present invention relates to means, methods, and uses that allow for contacting gametes with CO, inter alia, in a professional environment, such as a medical or ART laboratory, or a non-professional environment, such as a private environment. As such, the present invention also allows an individual, in particular a human with or without any medical experience/knowledge, to perform the means, methods, and uses of the present invention. The means and methods are thus suitable for home-use by an individual not familiar with or trained in artificial reproductive technology and/or any related field. It is evident, inter alia, from FIG. 1B, that home-use of the means and methods of the present invention comprises the collection of gametes in a container and activating a carbon monoxide releasing system (CORS). The activated CORS may be directly placed into the container and the container may be closed airtight. As has been described in detail herein above, the CORS may also be activated prior to the collection of gametes. In any event, gametes may be contacted with CO gas, ensuring their protection during liquification and subsequent handling of said gametes in assisted reproductive technologies (including cryopreservation). It is evident, inter-alia from FIG. 1B that a person not skilled in the art of artificial reproductive technology and/or any related field may be able to perform the means, methods, and uses of the present invention.

In the context of the present invention providing/obtaining gametes in a container and contacting said gametes with carbon monoxide, may refer to providing/obtaining the patients gametes in a container, providing/obtaining a carbon monoxide releasing system comprising a carbon monoxide releasing molecule, activating said carbon monoxide releasing system and/or molecule and thereby releasing carbon monoxide in said container, and contacting said gametes with carbon monoxide. In one aspect, said carbon monoxide releasing molecule may be obtained/provided in its activated form and, thus, does not require activation. In another aspect, said container may already contain carbon monoxide, a carbon monoxide releasing molecule and/or a carbon monoxide releasing system (as the one illustratively provided herein in form of the carbon monoxide releasing device/capsule).

The term “contacting/contacted” in context of the gametes/CO gas of the present invention relates to the physical touching of gametes and carbon monoxide gas. In one aspect, carbon monoxide gas may form a gas layer over said gametes. In some aspects, it may be necessary to actively mix gametes while the carbon monoxide gas is layered over the gametes in order to ensure even contacting of gametes with the carbon monoxide gas. Carbon monoxide may be provided to gametes as a solute, dissolved and/or solubilized in a liquid, the liquid being water, a water containing solution/suspension, including but not limited to bodily fluids, including but not limited to seminal fluid. It is evident to the skilled person, that the provision of CO gas to gametes or other tissues may result in CO becoming dissolved and/or solubilized in, for example, seminal fluid. Thus, it is evident that the term “CO gas” may also interchangeably refer to solubilized and/or dissolved CO. The provision of CO as a gas may be preferred whereby said provision can be directly (direct exposure to CO gas) or indirectly (for example by exposure to CO gas that is released from a corresponding carbon monoxide releasing molecule and/or carbon monoxide releasing system).

Carbon monoxide (CO) gas is a colorless, odorless, flammable gas that is slightly less dense than air which may make it necessary that the container may be lockable/sealable. CO consists of one carbon atom and one oxygen atom connected by a triple bond, which thus makes it the simplest molecule of the oxocarbon family. In coordination complexes (such as a metal carbonyl compound, which may, for example, be able to release CO upon the addition of a second compound such as FeCl₃ for example) the carbon monoxide ligand is termed carbonyl. CO has important biological roles across phylogenetic kingdoms. It is produced by many organisms, including humans. In mammalian physiology, carbon monoxide is a classic example of hormesis where low concentrations serve as an endogenous neurotransmitter (gasotransmitter) and high concentrations may be toxic resulting in carbon monoxide poisoning. In accordance with the present invention, this also makes it necessary that gametes may only be contacted with the inventive CO amounts as detailed herein below in order to prevent damaging the gametes.

As discussed herein, gametes in particular spermatozoa may be negatively influenced by the direct and/or indirect exposure to ROS/oxidants. Without being bound by theory, whenever the amount of oxidants exceeds the maximum capacity of the cellular redox buffer, oxidative stress may occur, inter alia, negatively affecting sperm motility. Conversely, whenever the amount of oxidants falls drastically below physiological levels, reductive stress may occur, inter alia, negatively affecting sperm motility. It is shown herein that the exposure of carbon monoxide to spermatozoa does not negatively influence their mobility. The successful exposure of gametes in particular spermatozoa of the present invention (exposure to CO with the herein described beneficial effects on said gametes) may comprise the inhibition or the reduction of undesired DNA fragmentation and/or the inhibition or the reduction of oxidation-reduction potential (like ROS). Also a maintenance or even an increase of gamete motility, in particular the motility of spermatozoa may be one of the effects of the present invention. In the appended examples corresponding technical details are provided how, inter alia, DNA fragmentation(s), ROS inhibition, and/or gamete motility/spermatozoa motility can be measured and assessed.

The skilled person is aware that reactive oxygen species (ROS) belong to the group of reactive molecular species (RMS), which further encompasses, inter alia, reactive nitrogen species (RNS) and reactive sulfur species (RSS). Since most RMS are highly reactive, they may react with each other which may result in their neutralization or in the emergence of new RMS (in biological cells, inter alia, in gametes, such as spermatozoa). In other words, ROS may react with, inter alia, other ROS, with RNS, and/or with RSS, which may result in the neutralization of said ROS or in the new emergence of ROS. In other words, the presence/emergence of ROS may result in the presence/emergence of more ROS and/or other RMS. Accordingly, in the context of the present invention it may be desired to reduce ROS levels/oxidative stress/oxidation-reduction potential in biological cells, inter alia, in gametes, such as spermatozoa in order to prevent/avoid/reduce the emergence/presence of (more) ROS and/or RMS. As, inter alia, illustratively shown in the appended Examples and Figures, the present invention provides for means and methods to effectively reduce oxidation-reduction potentials of biological cells/gametes/spermatozoa, which may, thus, prevent/avoid/reduce the emergence/presence of (more) reactive oxygen species.

Accordingly, within the herein provided means, methods, and uses gametes are preserved that are preferably to be used or intended to be used in reproductive technologies, like artificial reproductive technologies, in vitro fertilizations, preferably in mammals or in artificial insemination technologies, preferably of (farm) animals. Evidently, the “quality of the gametes” to be used in such reproductive technologies in humans as well as in animals is of utmost importance. As documented herein and as illustrated in the appended examples, the inventors have surprisingly found that exposure of gametes, in particular exposure of spermatozoa, to carbon monoxide can preserve and even improve said “quality of the gametes”. It is, inter alia, documented herein that exposure of gametes, in particular of spermatozoa, to carbon monoxide can abate and diminish disadvantageous effects of undesired oxidative processes, like the effects of reactive oxygen species (ROS). It is, inter alia, shown herein that the exposure of gametes (here spermatozoa) or of a (biological) sample comprising the gametes (like seminal fluid, ejaculate and/or a buffer/buffer system) to carbon monoxide leads to a surprising (yet desired) reduction of the oxidation-reduction potential of gametes. It is also shown that this contacting/exposure to/treatment with carbon monoxide preserves/maintains an already favorable (low) oxidation-reduction potential of gametes. Accordingly, the present invention provides the means, methods, and uses for obtaining gametes with a desired quality, for example for artificial reproductive technology or for (artificial) insemination technologies in which the quality of the gametes to be employed is of utmost importance. Accordingly, the present invention also relates to the provision of gametes useful in such technologies. The gametes (or a sample comprising said gametes) may be contacted with/exposed to the carbon monoxide in a container. Contacting of said gametes (or a sample comprising said gametes) in said container with carbon monoxide may result in the maintenance or increase of the quality of said gametes, i.e., in a preservation of the gametes. The provided gametes are to be contacted in the container with carbon monoxide gas for a sufficiently long time to ensure preservation of gametes. The term “for a sufficiently long time to ensure preservation of gametes” in context of this invention refers to the duration of contacting gametes and CO gas, which is necessary to keep gametes functional, alive, intact, or free from damage or decay after the gametes have been collected from a subject. Accordingly, the term “sufficiently long time” means an in vitro exposure of at least about 15 sec, at least 30 sec, at least 1 min, at least 5 min, at least 10 min, at least 30 min of the carbon monoxide to the seminal fluid/semen/ejaculate and/or manipulated corresponding samples like liquified seminal fluid/semen/ejaculate (this can be considered as the “exposure duration”). The relevant or desired exposure duration may, for example, be determined via measuring one or more characteristics of the CO-preserved gametes such as oxidation-reduction potential/ROS levels, DNA-fragmentation and/or motility (in case of sperms/spermatozoa), in particular progressive motility, by one or more methods as described in Examples 2 and 3 herein below and by comparing the results to results obtained using unpreserved gametes. In appended Examples 2 and 3, the inventors found that contacting of gametes with CO gas for 60 or 90 minutes may lead to CO-preserved gametes that exhibited (i) less oxidation-reduction-potential, (ii) less DNA fragmentation/DNA damage and (iii) a higher or maintained (progressive) motility compared to unpreserved gametes (i.e., gametes that were not contacted with CO) as is also evident, for example, from appended FIGS. 2 to 8. In the context of the present invention, contacting gametes with or to carbon monoxide may refer to a treatment with carbon monoxide and/or an exposure to carbon monoxide of said gametes for at least 15 min, at least 20 min, at least 30 min, at least 40 min, at least 50 min, at least 60 min, at least 70 min, at least 80 min, or at least 90 min. One of the non-limiting teachings of the invention is to provide gametes, preferably spermatozoa that are contacted with/exposed to/treated with carbon monoxide in order to remove/reduce negative influences of ROS (on said gametes). As is evident from the appended examples, the present inventors surprisingly found that contacting gametes, in particular spermatozoa with carbon monoxide provides the herein detailed beneficial effects in the preservation of said gametes, i.e. carbon monoxide positively influences and/or maintains the gamete quality. It is, inter alia, documented herein that these beneficial effects of carbon monoxide on gamete quality can even be shown after 90 min of carbon monoxide application in vitro, i.e., ex corpore.

In a preferred embodiment of the present invention, carbon monoxide (CO) gas may be released from a carbon monoxide releasing molecule (CORM). In one aspect, the CORM which may be located inside of a closed compartment (such as a capsule, for example, that is only permeable for CO-gas) may be added to the container (comprising the gametes). Accordingly, the CORM may be not in direct contact with the gametes. The CORM may preferably be a metal carbonyl compound, even more preferably a molybdenum carbonyl compound and most preferably trisodiumtricarbonyl-[tris(isocyanoethylacetate)]molybdenum (chemical formula: Na₃Mo(CO)₃ (CNCH₂CO₂H)₃). NasMo(CO)₃(CNCH₂CO₂H)₃ may in the following also be termed Mo-CORM.

Carbon monoxide releasing molecules that are suitable for use in the present invention have been described in WO 2015/188941 A1, WO 2016/110517 A1 and DE 10 2017 006 393 A1, all of which are incorporated herein by reference.

Preferably, the carbon monoxide releasing molecule (CORM) is a metal carbonyl compound. The metal carbonyl compound may comprise, e.g., a complex of an element of the group of Rh, Ti, Os, Cr, Mn, Fe, Co, Mo, Ru, W, Re, Ir, B and C. More preferably, the metal carbonyl compound may comprise a complex of an element of the group of Rh, Mo, Mn, Fe, Ru, B and C, even more preferably of the group of Rh, Fe, Mn, Mo, B and C. The metal carbonyl compounds may be regarded as complexes, because they comprise CO groups coordinated to a metal center. However, the metal may be bonded to other groups by other than coordination bonds, e.g., by ionic or covalent bonds. Thus, groups other than CO, which form part of the metal carbonyl compound, need not strictly be “ligands” in the sense of being coordinated to a metal center via a lone electron pair, but are herein referred to as “ligands” for ease of reference.

Thus, the ligands to the metal may all be carbonyl ligands. Alternatively, the carbonyl compound may comprise at least one ligand which is not CO. Ligands which are not CO may be typically neutral or anionic ligands, such as halide, or derived from Lewis bases and have N, P, O or S or a conjugated carbon group as the coordinating atom(s). Preferred coordinating atoms may be N, O and S. Examples include, but are not limited to, sulfoxides such as dimethylsulfoxide, natural and synthetic amino acids and their salts for example, glycine, cysteine, and proline, amines such as NEt₃ and H₂NCH₂CH₂NH₂, aromatic bases and their analogues, for example, bi-2,2′-pyridyl, indole, pyrimidine and cytidine, pyrroles such as biliverdin and bilirubin, drug molecules such as YC-1 (2-(5′-hydroxymethyl-2′-furyl)-1-benzylindazole), thiols and thiolates such as EtSH and PhSH, chloride, bromide and iodide, carboxylates such as formate, acetate, and oxalate, ethers such as Et₂O and tetrahydrofuran, alcohols such as EtOH, and nitriles such as MeCN. Other possible ligands are conjugated carbon groups, such as dienes, e.g., cyclopentadiene (C₅H₅) or substituted cyclopentadiene. The substituent group in substituted cyclopentadiene may be, for example, an alkanol, an ether, or an ester, e.g., —(CH₂)ₙOH where n may be 1 to 4, particularly —CH₂OH, —(CH₂)ₙOR where n may be 1 to 4 and R may be hydrocarbon preferably alkyl of 1 to 4 carbon atoms and —(CH₂)ₙOOCR where n may be 1 to 4 and R may be hydrocarbon preferably alkyl of 1 to 4 carbon atoms. The preferred metal in such a cyclopentadiene or substituted cyclopentadiene carbonyl complex may be Fe.

For a detailed description of carbon monoxide releasing compounds, it is also explicitly referred to WO 2008/130261 A1 and US 2007/0219120 A1 which are incorporated herein by reference. There, aldehydes according to formula I

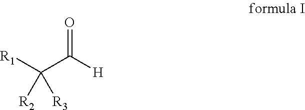

are disclosed which may also be used as CORM in the present invention wherein R₁, R₂ and R₃ are each independently selected from alkyl, substituted alkyl, cycloalkyl, substituted cycloalkyl, heterocyclyl, substituted heterocyclyl, alkylheterocyclyl, substituted alkylheterocyclyl, alkenyl, substituted alkenyl, aryl, substituted aryl, heteroaryl, substituted heteroaryl, alkylaryl, substituted alkylaryl, wherein the number of C atoms may be 1-12 or 1-6 in each case hydroxy, alkoxy, amino, alkylamino, mercapto, alkylmercapto, aryloxy, substituted aryloxy, heteroaryloxy, substituted heteroaryloxy, alkoxycarbonyl, acyl, acyloxy, acylamino, alkylsulfonyl, alkylsulfinyl, F, Cl, Br, NO₂ and cyano; or two or more of R₁, R₂ and R₃ may be taken together to form a substituted or unsubstituted carbocyclic or heterocyclic ring structure or an derivative thereof. For any substituent the number of C atoms may be 1-12 or 1-6.

A derivative of a compound of formula I being an acetal, hemiacetal, aminocarbinol, aminal, imine, enaminone, imidate, amidine, iminium salt, sodium bisulfite adduct, hemimercaptal, dithioacetal, 1,3-dioxepane, 1,3-dioxane, 1,3-dioxalane, 1,3-dioxetane, α-hydroxy-1,3-dioxepane, α-hydroxy-1,3-dioxane, α-hydroxy-1,3-dioxalane, α-keto-1,3-dioxepane, α-keto-1,3-dioxane, α-keto-1,3-dioxalane, α-keto-1,3-dioxetane, macrocyclic ester/imine, macrocyclic ester/hemiacetal, oxazolidine, tetrahydro-1,3-oxazine, oxazolidinone, tetrahydro-oxazinone, 1,3,4-oxadiazine, thiazolidine, tetrahydro-1,3-thiazine, thiazolidinone, tetrahydro-1,3-thiazinone, imidazolidine, hexahydro-1,3-pyrimidine, imidazolidinone, tetrahydro-1,3-pyrimidinone, oxime, hydrazone, carbazone, thiocarbazone, semicarbazone, semithiocarbazone, acyloxyalkyl ester derivative, O-acyloxyalkyl derivative, N-acyloxyalkyl derivative, N-Mannich base derivative or N-hydroxymethyl derivative may also be used as CORM in the present invention.

The CORM of the present invention may e.g., also be trimethylacetaldehyde, 2,2-dimethyl-4-pentenal, 4-ethyl-4-formyl-hexanenitrile, 3-hydroxy-2,2-dimethylpropanal, 2-formyl-2-methyl-propylmethanoate, 2-ethyl-2-methyl-propionaldehyde, 2,2-dimethyl-3-(p-methylphenyl) propanal or 2-methyl-2-phenylpropionaldehyde.

In one aspect, an oxalate, an oxalate ester, or amide may be used as CORM in accordance with the present invention.

Preferred CORMs for use in the present invention may comprise molybdenum carbonyl compounds, CORM-1, CORM-2, CORM-3, CORM-401, as e.g., disclosed in WO 2015/188941 A1, WO 2016/110517 A1 and DE 10 2017 006 393 A1.

More preferred in context of this invention may be molybdenum-based CORMs, such as Mo(CO)₃(CNC(CH₃)₂COOH)₃ (also designated as “CORM-ALF794”) and Mo(CO)₃(CNCH₂CO₂H)₃(trisodiumtricarbonyl-[tris(isocyanoethylacetate)]molybdenum), wherein Mo(CO)₃(CNCH₂CO₂H)₃ may be particularly preferred. Preferably, the tri-sodium salt (Na₃Mo(CO)₃(CNCH₂CO₂)₃) (“Mo-CORM”) may be used.

Whenever a CORM is used in context of the means, methods, and uses of the present invention, the CORM is to be contacted with a second compound thereby releasing the carbon monoxide gas. A second compound may be FeCl₃, Ce(SO₄)₂ or H₂O₂. In a preferred embodiment of the present invention, the second compound may be FeCl₃.

Second compounds suitable for use in the present invention are also described in WO 2015/188941 A1, WO 2016/110517 A1 and DE 10 2017 006 393 A1, all of which are incorporated herein by reference.

The CO-releasing molecule (CORM) releases carbon monoxide gas upon contact with the second compound. “Contact” in this context means that a reaction between the CORM and the second compound may take place, which results in CO gas release. Upon contact with the second compound, the CORM starts to release (substantial amounts of) CO gas as will be further detailed herein below. The system (i.e., the CORS) is then “activated” (activated CORS), and the released CO gas may exert its gamete-preserving activity/function.

The second compound may be a sulfur-containing compound, a nitrogen-containing compound, an oxidizing compound, an acid or a base, or water.

When the CORM is a metal carbonyl compound, the second compound may, e.g., be a carbonyl substituting agent, such a sulfur-containing compound or a nitrogen-containing compound. The sulfur-containing compound may, e.g., be selected from an alkali metal or alkaline-earth metal salt, preferably a sodium salt of sulfite, dithionite, or metabisulfite, or a compound bearing at least one thiol moiety, such as cysteine or glutathione.

Examples of oxidizing compounds which may be used as second compounds in the means, methods, and uses of the present invention include peroxides, perborates, percarbonates, and nitrates of which calciumperoxide, dibenzoylperoxide, hydrogen peroxide urea, sodium perborate, and sodium percarbonate may be preferred. Oxidizing metal salts that may be used as second compounds may include silver(I)nitrate, iron(III)chloride, potassium permanganate, cer(IV)sulfate, potassium dichromate, gold(III)chloride and silver nitrate, wherein iron(III)chloride, potassium permanganate and cer(IV)sulfate and, in particular, iron(III)chloride and cer(IV)sulfate, may be preferred. The oxidizing metal salts may preferably be used in aqueous solution, as is also evident from the appended examples.

In terms of acids, e.g., hydrogen chloride (HCl) may be used. In another embodiment, the second compound may be a non-enzymatic compound. Preferably, the second compound may be a compound with a molecular weight of less than 10,000 g/mol, more preferably of less than 7,000 g/mol or even less than 1,000 g/mol. The second compound may, e.g., also be water or a solvent. A preferred CORM which releases carbon monoxide gas upon contact with water may be ALF186.

If a metal carbonyl compound as CORM is used together with a sulfur-containing compound or other electron withdrawing compound as the second compound, it is e.g., believed that, when this second compound comes into contact with the metal carbonyl compound, a ligand substitution takes place, thereby triggering CO gas release.

In another embodiment of the present invention, the second compound may be selected from the group consisting of a sulfur-containing compound, a nitrogen-containing compound, an oxidizing compound, and water. This may in particular be the case if the CORM is a metal carbonyl compound.

In preferred embodiments of the present invention, molybdenum carbonyl compounds may be used as CORM and oxidizing compounds may be used as the second compound. In a further preferred alternative preferred embodiment, Ru₂(CO)₆Cl₄ may be used as CORM and sodium sulfite (Na₂SO₃) may be used as second compound.

Particularly preferred embodiments of the present invention may include combinations of a molybdenum carbonyl compound, preferably Mo(CO)₃(CNCH₂COOH)₃ or its Na₃Mo(CO)₃(CNCH₂CO₂H)₃ tri-sodium salt (Mo-CORM) together with iron(III)chloride (FeCl₃), cer(IV)sulfate (Ce(SO₄)₂) or H₂O₂, wherein FeCl₃ and Ce(SO₄)₂ are used in one embodiment as aqueous solutions at concentrations between about 2 and about 3 mol/L, and H₂O₂ is used as aqueous solution at a concentration of about 20 to 40 wt.-%, preferably about 30 wt.-%. Molybdenum carbonyl compounds may have the advantage of producing CO at a high capacity (>95%) and with a high purity (>95%). For achieving a particularly high CO production with a particularly high purity, it may be particularly preferred to use Mo(CO)₃(CNCH₂COOH)₃ or its Na₃Mo(CO)₃(CNCH₂CO₂H): tri-sodium salt (Mo-CORM) in combination with FeCl₃.

The second compound may also be added to the container as part of the closed compartment (such as a capsule) which also comprises the CORM. Therefore, in a preferred embodiment, CORM and said second compound may together be comprised in one closed compartment such as a capsule which may be added to the inside of the container containing the gametes. Such a system comprising CORM, and a second compound inside of a closed compartment may also be termed a CO-releasing system (“CORS”). The outer sheath of such a CORS may be selectively permeable for gaseous molecules, in particular for CO gas. This also ensures that all other components comprised in the CORS remain inside the CORS/the corresponding closed compartment (capsule) and thus are not contacted to the gametes. In one aspect, CORM and the second compound may be physically separated within the closed compartment (for example by a separating membrane or a septum). Only the physical contact of CORM and the second compound releases CO from the CORM/CORS (“activated CORS”) by diffusion of CO gas through the outer sheath into the container containing the gametes. As is also evident from appended Example 2 below, the inventors have used in one exemplified embodiment one CORS-capsule (18-22 mm in length and 6-8 mm diameter) containing 15 mg of Mo-CORM and 150 μl of an aqueous FeCl₃ solution (583.3 mg/ml) separated from each other by a septum/membrane to preserve human sperm samples after liquefaction. By applying pressure on the longitudinal axis of the CORS/closed compartment/capsule, CORM and FeCl₃ are brought into physical contact to generate enough CO gas in a gas-tight 15 mL Falcon tube over the course of 90 minutes to ensure preservation of gametes.

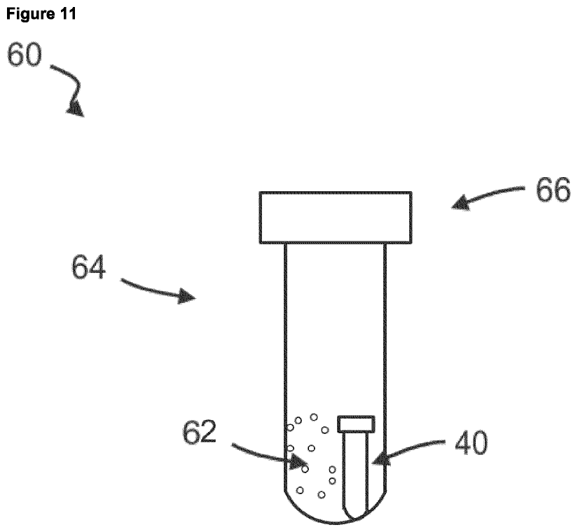

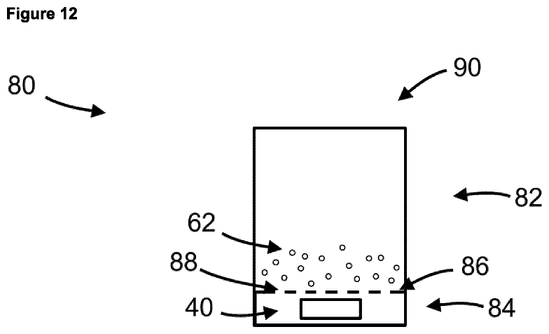

In the context of this invention, especially in the herein described in vitro and or ex corpore methods, also CO releasing molecules (CORM) like trisodiumtricarbonyl-[tris(isocyanoethylacetate)]molybdenum maybe used. Also, carbon monoxide releasing systems may be employed. A corresponding, yet illustrative, carbon monoxide releasing system (CORS) is show in appended FIGS. 11 and 12. In these figures a novel and inventive CORS is provided in form of a CORS capsule as also disclosed and described in EP22216317.2 and in PCT/EP2023/074808. This CORS-capsule is also illustrated in the device with the reference sign “40” in FIGS. 11 and 12. The terms “CORS”, “carbon monoxide releasing device” and “carbon monoxide releasing system” may be used interchangeably in context of the present invention. The CORS may be configured for treatment of biological cells, preferably living cells, ex vivo, by releasing the carbon monoxide ex vivo. A corresponding configuration is illustrated under reference sign “60” in FIG. 11. “62” illustrates biological cells, like gametes, in particular spermatozoa (as also comprised in corresponding seminal fluid/semen/ejaculate). The system “60” may be configured for treating biological cells “62”, preferably living cells, preferably gametes, with carbon monoxide. The system “60” may be configured to provide one or more effects on the biological cells “62” which at least partially preserve the biological cells “62”. The system “60” may include at least one container “64” configured to receive the biological cells “62” and at least one source of carbon monoxide. The container “64” and the source of the CORS “40” may be arranged relative to each other such that the biological cells “62” may be contacted by the carbon monoxide provided by the CORS “40” to treat the biological cells “62”. The container “64” may include a lid “66”, which is preferably configured to provide a seal with the container “64” to seal the contents of the container “64” from an environment, preferably in a gas-tight manner. Alternatively, or additionally, the treatment system “60” may be configured for therapeutically treating an animal body and/or a human body ex vivo, e.g., by applying carbon monoxide to an outer surface, e.g., the skin, of the animal body and/or the human body, which may also be considered as treating biological cells within the meaning of the present disclosure.

FIG. 12 shows, in a schematic view, an alternative system to be used may be the one illustrated in “80” of said figure. Also “80” can be used for exposing biological, living cells, preferably gametes, ex vivo, to carbon monoxide as provided herein. Said exposure may comprise releasing of the carbon monoxide in an ex vivo context. The system “80” may include at least one container “82” configured to receive the biological cells “62” and at least one source of carbon monoxide, which in the configuration shown in FIG. 12 is embodied, as an example but not limited thereto, by the CORS “40”. The container “82” and the source of carbon monoxide device “40” may be arranged relative to each other such that the biological cells “62” may be contacted by the carbon monoxide provided by the device “40” to contact the biological cells “62” with carbon monoxide. The container “82” may include at least one compartment “84” configured to receive, and preferably house and/or secure, preferably in a captive manner, the device “40”. Alternatively, or in addition to the compartment “84”, the container “82” may include one or more securing means, e.g., one or more clips, configured to secure, preferably captively and/or substantially immovably, the CORS “40” in the container “82”. This may allow the system “80” to be preassembled, in particular by arranging the CORS “40” in the compartment “84” prior to use, preferably prior to being distributed to the locations of application, e.g., one or more laboratories or medical institutions and the like. This may facilitate handling and/or use of the system “80”.

Corresponding systems for the exposure of CO to gametes are illustrated and provided in EP22216317.2 and in PCT/EP2023/074808 which are incorporated herewith by reference. The CO gas may also be directly supplied to the container via a separate tube that releases the CO gas into the container. In one aspect, CO gas may be directly supplied from a pressurized CO gas tank, or it may be released from liquids like saturated solutions, foams, hydrogels, or solids where CO is physically bound.

The total amount of CO gas applied to the gametes in the container may be between about 20 μmol and about 500 μmol, preferably about 30 μmol and about 450 μmol, about 40 μmol and about 400 μmol, preferably between about 45 μmol and about 270 μmol, preferably between about 60 μmol and about 180 μmol. The term “about” followed by a value in this context refers to the value itself but also includes a margin (of error) of ±10% of that value. For illustration, 1 mg of Mo-CORM may be able to release about 6 μmol of carbon monoxide gas if each of the Mo-CORM molecules gets decarbonylated completely. As is also illustrated in appended non-limiting Example 2, in the case of human spermatozoa samples of about 0.7 to 2.9 ml volume inside a 15 ml volume standard Falcon tube 15 mg of Mo-CORM may be used for preservation. This amount corresponds to 15×6 μmol=90 μmol of released carbon monoxide gas. For further illustration, in case of cattle from which sperm samples with a higher average volume may be collected (5-8 mL), four times the amount of Mo-CORM, i.e., 60 mg of MO-CORM may be used. This amount corresponds to a maximum release of 360 μmol of carbon monoxide. Hence, the amount of Mo-CORM/CO to be used to contact gametes may also depend on the volume of the collected gametes and the volume of the container. In one exemplified embodiment, i.e., in Example 2 an average of about 8.4 mg CORM per mL of collected sample was used (range: about 5.2 mg to 20.5 mg CORM per mL of collected sample).

In accordance with the means, methods, and uses of the present invention, gametes may be contacted with carbon monoxide gas at about 25 to about 35 °C for about 15 minutes to about 120 minutes, preferably for about 30 minutes to about 90 minutes, more preferably for about 30 minutes to about 60 minutes to ensure preservation of gametes. The term “about” followed by a value in this context again refers to the value itself but also includes a margin (of error) of ±10% of that value.

After the gametes have been contacted in the container with carbon monoxide gas, there are several methods which may be used to determine if gametes have been successfully preserved, some of which are also described in the appended Example 2. Preservation of gametes in accordance with the present invention may be assessed via the determination of (spermatozoon/sperm cell) motility, in particular progressive (spermatozoon/sperm cell) motility, DNA-fragmentation, spermiogram, oxidation-reduction potential measurement(s) or a combination thereof. As is also detailed herein above and below, the Male Infertility Oxidative System (MiOXSYS, Englewood, CO) for example may be used to determine (static) oxidation-reduction potential/ROS levels of spermatozoa. It is evident for the person skilled in the art that the terms static oxidation-reduction potential, oxidation-reduction potential, ROS levels, oxidative potential, and oxidative-reductive potential may be used interchangeably in the context of the present invention. The Halo Sperm G2 Kit (Halotech, Madrid, Spain) may be used to determine DNA-fragmentation of spermatozoa and the CEROS II system (Hamilton Thorne, Beverly, MA) may be used to determine sperm motility, in particular total and progressive motility. A spermiogram (also referred to as seminogram or semen analysis) on the other hand may analyze one or more than one characteristic(s) of a male's semen and sperm cells contained therein. Depending on the measurement kit/method, just a few characteristics may be evaluated (such as with home kits) or many characteristics may be evaluated simultaneously (for example by a diagnostic laboratory). Non-limiting characteristics to be analyzed in context of a spermiogram may, inter alia, comprise physical characteristics of semen (color, odor, pH, viscosity, and liquefaction), volume, sperm number, concentration, morphology, sperm total motility and progression/progressive motility, non-progressive motility, percentage of immotile sperm, percentage of vital sperm, and percentage of normal forms of sperm. A person skilled in the art is aware of the multitude of commercially available kits and means and methods to analyze the above characteristics.

All of the above explanations which have been described in context of the means and methods of the present invention also apply mutatis mutandis to the following uses, methods of treatment and kits which are detailed in the following.

Thus, in another embodiment, the present invention also relates to the use of carbon monoxide gas for the preservation of gametes for reducing the risk of congenital abnormality and/or aneuploidy (and/or euploidy) in an assisted reproductive technology (application). To that end, said gametes are contacted with carbon monoxide gas in a container for a sufficiently long time to ensure preservation of gametes.

In another embodiment, the present invention relates to a method for reducing DNA-fragmentation and/or oxidation-reduction potential of gametes in an assisted reproductive technology, wherein said gametes are contacted with carbon monoxide gas. Said gametes may be obtained/provided in a container and contacted with CO in said container. Said gametes may be contacted with CO for a sufficiently long time (to ensure a reduction of DNA-fragmentation and/or oxidation-reduction potential of the gametes). Here, reducing DNA-fragmentation and/or oxidation-reduction potential of gametes may maintain and/or increase motility including but not limited to progressive motility, total motility, and/or VAP of said gametes. Further, reducing DNA-fragmentation and/or oxidation-reduction potential of gametes may reduce the risk of congenital abnormality and/or aneuploidy in an artificial reproductive technology. In another aspect, reducing DNA-fragmentation and/or oxidation-reduction potential of gametes may improve the success/success-rates of an artificial reproductive technology. Accordingly, reducing DNA-fragmentation and/or oxidation-reduction potential of gametes may reduce the risk of miscarriage and/or pregnancy loss in an artificial reproductive technology.

In another embodiment, the present invention relates to the use of carbon monoxide in reducing fragmentation and/or oxidation-reduction potential of gametes in an assisted reproductive technology, wherein said gametes are contacted with carbon monoxide gas. Said gametes may be obtained/provided in a container and contacted with CO in said container. Said gametes may be contacted with CO for a sufficiently long time to ensure a reduction of DNA-fragmentation and/or oxidation-reduction potential of the gametes. Here, reducing DNA-fragmentation and/or oxidation-reduction potential of gametes may maintain and/or increase motility including but not limited to progressive motility, total motility, and/or VAP of said gametes. Further, reducing DNA-fragmentation and/or oxidation-reduction potential of gametes may reduce the risk of congenital abnormality and/or aneuploidy in an artificial reproductive technology. In another aspect, reducing DNA-fragmentation and/or oxidation-reduction potential of gametes may improve the success/success-rates of an artificial reproductive technology. Accordingly, reducing DNA-fragmentation and/or oxidation-reduction potential of gametes may reduce the risk of miscarriage and/or pregnancy loss in an artificial reproductive technology.

In another embodiment the present invention also relates to carbon monoxide for use in treating/preventing gamete-related diseases caused by/linked to ROS/elevated ROS levels.

As already detailed herein above and below, reactive oxygen species and/or oxidative stress (and as such also elevated oxidation-reduction potential) can cause/be linked to congenital abnormality and/or aneuploidy in the progeny resulting from a diseased gamete and/or a gamete with high ROS-levels. As such, contacting gametes with CO gas may prevent/reduce DNA fragmentation and/or oxidation reduction potential and may consequently prevent and/or reduce the risk for congenital abnormality and/or aneuploidy (in an artificial reproductive technology). Hence, contacting gametes with CO gas may prevent and/or reduce the risk for congenital abnormality and/or aneuploidy. In other words, the present invention relates to carbon monoxide for use in preventing abnormality and/or aneuploidy. In another aspect, the present invention relates to carbon monoxide for use in a method of preventing abnormality and/or aneuploidy. It is evident, inter alia, from the Examples that gametes may be (provided/obtained in a container and) contacted with CO gas (in said container) ex vivo (for a sufficiently long time). However, the present invention also relates to contacting gametes with CO in vivo (for a sufficiently long time), thereby, preventing and/or reducing the risk for congenital abnormality and/or aneuploidy, as is described in more details herein below.

As already detailed herein above and below, reactive oxygen species and/or oxidative stress (and as such also elevated oxidation-reduction potential) of gametes can cause/be linked to male infertility in a patient. Thus, it is evident to the person skilled in the art that depleting and/or reducing reactive oxygen species, oxidative stress and/or oxidation-reduction potential in gametes of a male patient may treat/reduce infertility of said patient. In other words, the present invention relates to carbon monoxide for use in treating/reducing male infertility. In another aspect, the present invention relates to carbon monoxide for use in a method of treating and/or reducing male infertility. It is evident, inter alia, from the Examples that gametes may be (provided in a container and) contacted with CO (gas in said container) ex vivo (for a sufficiently long time). However, the present invention also relates to contacting gametes with CO in vivo (for a sufficiently long time), thereby treating and/or reducing male infertility, as is described in more details herein below.

In another aspect, reactive oxygen species and/or oxidative stress (and as such also elevated oxidation-reduction potential) and DNA-fragmentation of gametes can cause and/or is linked to diseases. Such diseases may be caused by and/or linked to (elevated) (physiological and/or psychological) stress levels in a patient. Such diseases may occur in a patient suffering from (physiological and/or psychological) stress. Contacting gametes with CO gas may prevent and/or reduce DNA fragmentation and/or oxidation reduction potential and may consequently prevent/treat such diseases (in an artificial reproductive technology). Hence, contacting gametes with CO gas may prevent and/or treat such diseases. In other words, CO may treat and/or prevent a disease caused by and/or linked to (elevated) DNA fragmentation and/or oxidation-reduction potential of gametes. In other words, the present invention relates to carbon monoxide for use in treating and/or preventing a disease caused by and/or linked to (elevated) DNA fragmentation and/or oxidation-reduction potential of gametes. In another aspect, the present invention relates to carbon monoxide for use in a method of treating and/or preventing a disease caused by and/or linked to (elevated) DNA fragmentation and/or oxidation-reduction potential of gametes. It is evident, inter alia, from the Examples that gametes may be (provided/obtained in a container and) contacted with CO gas (in said container) ex vivo (for a sufficiently long time). However, the present invention also relates to contacting gametes with CO in vivo (for a sufficiently long time), thereby, preventing and/or treating such diseases, as is described in more details herein below.

In another aspect, elevated ROS levels can cause and/or be linked to diseases of gametes. Such elevated ROS levels and thereby such diseases may be caused by/linked to elevated (physiological and/or psychological) stress levels in a patient. Such diseases may occur in a patient suffering from (physiological and/or psychological) stress. Contacting gametes with CO gas may reduce (elevated) ROS levels and may consequently prevent and/or treat such diseases (in an artificial reproductive technology). In other words, CO may treat and/or prevent a disease of gametes caused by and/or linked to elevated ROS levels. In other words, the present invention relates to carbon monoxide for use in treating and/or preventing a disease of gametes caused by and/or linked to elevated ROS levels. In another aspect, the present invention relates to carbon monoxide for use in a method treating and/or preventing a disease of gametes caused by and/or linked to elevated ROS levels. It is evident, inter alia, from the Examples that gametes may be (provided/obtained in a container and) contacted with CO gas (in said container) ex vivo (for a sufficiently long time). However, the present invention also relates to contacting gametes with CO in vivo (for a sufficiently long time), thereby, preventing and/or treating such diseases, as is described in more details herein below.

The present invention further relates to carbon monoxide for use in treating and/or preventing pregnancy loss and/or miscarriage caused by (elevated) ROS and/or (elevated) DNA fragmentation (in a gamete/gametes used for fertilization).

It is further evident that reducing DNA-fragmentation and/or oxidation reduction potential of gametes may improve the success and/or success-rates of artificial reproductive technologies using said gametes for fertilization. In another aspect, reducing DNA-fragmentation and/or oxidation reduction potential of gametes may protect said gametes and/or prevent damaging said gametes. In further aspect, reducing DNA-fragmentation and/or oxidation reduction potential of gametes may increase viability of said gametes. In another aspect, reducing DNA-fragmentation and/or oxidation reduction potential of gametes may maintain and/or improve (total) motility of said gametes. In a further aspect, reducing DNA-fragmentation and/or oxidation reduction potential of gametes may maintain and/or improve progressive motility of said gametes. In another aspect, reducing DNA-fragmentation and/or oxidation reduction potential of gametes may maintain and/or improve average path velocity (VAP) of said gametes.

As has already been detailed herein above, preservation of gametes with carbon monoxide (gas) in a functional, alive, viable, intact state or a state free from damage or decay reduces the risk of congenital abnormality and/or aneuploidy. It is evident for the person skilled in the art, that the terms “functional”, “alive”, and “viable” as used herein are interchangeable with regard to gametes. As such, improving and/or increasing sperm and/or spermatozoan viability, quality, and/or viability parameters may be used interchangeably in the context of the present invention. This is in particular the case, when CO-preserved gametes are used in an assisted reproductive technology application. Thus, the term “reducing the risk” refers to the lowered risk that is associated with using CO-preserved functional/alive/intact gametes in an assisted reproductive technology (application) as compared to using unpreserved gametes which may be more damaged and thus lead to, for example, a newborn who suffers from congenital abnormality or aneuploidy with a higher likelihood. Accordingly, contacting/exposing gametes, specifically spermatozoa, with/to carbon monoxide/carbon monoxide gas can preserve functional/alive/intact gametes with (desired) maintained or even improved quality, viability, and/or viability parameters. In the context of the present invention, said carbon monoxide-preserved gametes, when used, inter alia but not limiting in an artificial reproductive technology may also reduce the risk of miscarriage of a zygote, an embryo, a progeny, etc. derived from one of said preserved gametes, as compared to a gamete that was not preserved/contacted with/exposed to said carbon monoxide. In other words, contacting gametes, specifically spermatozoa with carbon monoxide (gas), may increase success/success rates of an artificial reproduction technology using said preserved gamete as compared to a gamete not treated with carbon monoxide (gas).

Assisted reproductive technology (ART) comprise medical procedures used primarily to address infertility (which is often a consequence of non-functional/dead/damaged gametes) and to reduce the risk of congenital abnormality or aneuploidy. Non-limiting examples of ART applications in accordance with the present invention may, inter alia, comprise in vitro fertilization (IVF), intracytoplasmic sperm injection (ICSI), intrauterine insemination (IUI), frozen embryo replacement (FER), preimplantation genetic testing (PGT; which may be done in addiction to IVF), in vitro maturation of oocytes (IVM), cryopreservation of gametes, frozen oocyte replacement (FOR), gamete intrafallopian transfer (GIFT), zygote intrafallopian transfer (ZIFT), or cryopreservation, some of which are also described in Wyns, Human Reproduction Open 3 (2021): 1-17.

Congenital abnormalities comprise a wide range of abnormalities of body structure or function that are present at birth and are of prenatal origin. For efficiency and practicality, the focus is commonly on major structural abnormalities. These are defined as structural changes that have significant medical, social, or cosmetic consequences for the affected individual, and typically require medical intervention.

Thus, congenital abnormality/abnormalities in accordance with the present invention may inter alia be selected from the group consisting of congenital abnormality of the limb, congenital abnormality of the heart, congenital abnormality of the nervous system, congenital abnormality of the gastrointestinal system, and congenital abnormality of the lung.

Congenital abnormality of the limb in accordance with the present invention may inter alia be selected from the group consisting of achondroplasia, amelia, amniotic band syndrome, brachydactyly, cleidocranial dysostosis, congenital aplasia or hypoplasia, ectrodactyly, oligodactyly, phocomelia, polydactyly, polymelia, polysyndactyly, and syndactyly.

Congenital abnormality of the heart in accordance with the present invention may inter alia be selected from the group consisting of atrial septal defect, patent ductus arteriosus, tetralogy of fallot and ventricular septal defect.

Congenital abnormality of the nervous system in accordance with the present invention may inter alia be selected from the group consisting of neural tube defects such as agenesis of the corpus allosum, anencephaly, Arnold-Chiari malformation, Dandy-Walker malformation, encephalocele, holoprosencephaly, hydrocephalus, lissencephaly, megencephaly, meningocele, meningomyelocele, microencephaly, polymicrogyria, and spina bifida. Congenital abnormality of the gastrointestinal system in accordance with the present invention may inter alia be selected from the group consisting of atresia, imperforate and stenosis. Congenital abnormality of the lung in accordance with the present invention may inter alia be congenital bronchiectasis.

(Congenital) Aneuploidy in context of the present invention relates to the presence of an abnormal number of chromosomes in a cell, for example a human cell having 45 or 47 chromosomes instead of the usual 46. It does not include a difference of one or more complete sets of chromosomes. Aneuploidy originates during cell division when the chromosomes do not separate properly between the two cells (nondisjunction). Most cases of aneuploidy in the autosomes result in miscarriage, and the most common extra autosomal chromosomes among live births are 21, 18 and 13 (Driscoll, The New England Journal of Medicine 360 (2009): 2556-2562). Chromosome abnormalities are detected in 1 of 160 live human births. Non-limiting examples of (congenital) aneuploidy may, inter alia, be selected from the group consisting of trisomy 1 (1p36 deletion syndrome/1q21.1 deletion syndrome), trisomy 2 (2q37 deletion syndrome), trisomy 3, trisomy 4 (Wolf-Hirschhorn syndrome), trisomy 5 (Cri du chat/5q deletion syndrome), trisomy 6, trisomy 7 (Williams syndrome), trisomy 8 (Monosomy 8p/Monosomy 8q), trisomy 9 (Alfi's syndrome/Kleefstra syndrome), trisomy 10 (Monosomy 10p/Monosomy 10q), trisomy 11 (Jacobsen syndrome), trisomy 12, Patau syndrome, trisomy 14, trisomy 15 (Angelman syndrome/Prader-Willi syndrome), trisomy 16, trisomy 17 (Miller-Dieker syndrome/Smith-Magenis syndrome), Edwards syndrome (Distal18q-/Proximal 18q-, trisomy 19, trisomy 20, trisomy 21 (Down syndrome), Cat eye syndrome/trisomy 22 (DiGeorge syndrome/Phelan-McDermid syndrome/22q11.2 distal deletion syndrome).

(Congenital) Euploidy in context of the present invention relates to a cell with any number of complete chromosome sets (“sets”) that is different from a chromosome set of 2 which is contained in normal diploid cells. Non-limiting examples of euploid cells with a different number of complete chromosome sets that is different from a chromosome set of two may inter alia comprise monoploid (1 set), triploid (3 sets), tetraploid (4 sets), pentaploid (5 sets), hexaploid (6 sets), heptaploid/septaploid (7 sets) cells. The generic term polyploid may often be used to describe cells with three or more chromosome sets.

In one further embodiment, the present invention relates to a method for treating congenital abnormality or aneuploidy (or euploidy) comprising contacting carbon monoxide gas to the gametes of a patient in need thereof. The contacting of gametes with carbon monoxide may, for example, be done according to the method as described herein above.

Hence, in order to treat congenital abnormality or aneuploidy (or euploidy) as described above with carbon monoxide gas, it may be necessary to diagnose that a healthy or diseased patient or a patients' gametes may be in need of such a treatment prior to their use in ART for example. A patient or a patients' gametes may be in need of such treatment if already existing offspring of said patient suffer(s) from congenital abnormality or aneuploidy (or euploidy) or if a patients' gametes are assessed to have a higher risk of leading to congenital abnormality or aneuploidy (or euploidy) as compared to gametes of a normal control patient (cohort). In the context of the present invention, the terms “patient”, “diseased patient”, “patient in need of such treatment”, “diseased subject”, “individual”, and “individual to be treated” may be used interchangeably and may refer to a subject having gametes with increased DNA fragmentation and/or oxidation-reduction potential.

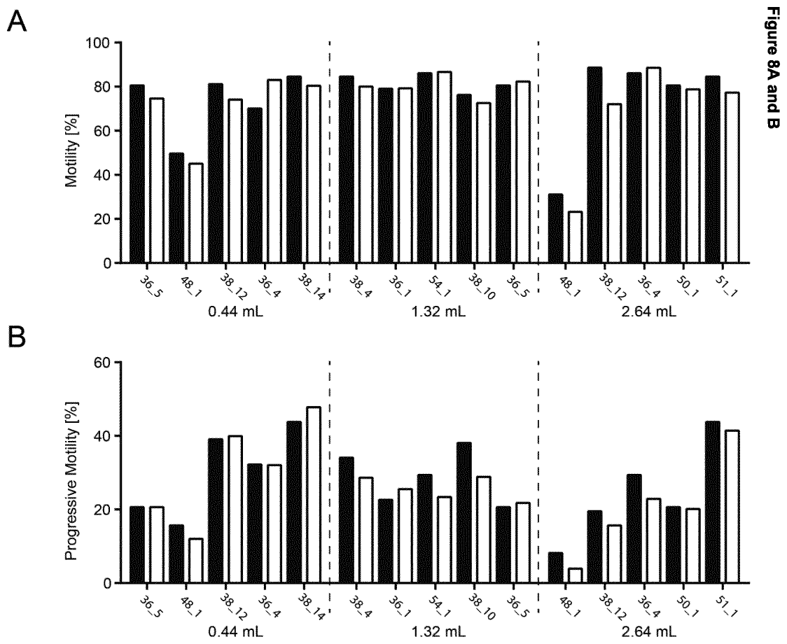

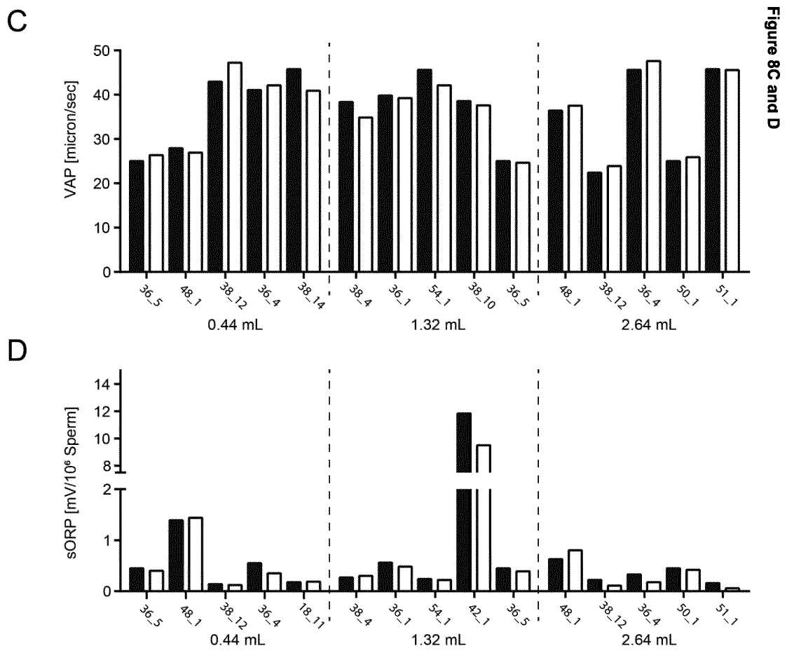

Such a gamete assessment may, inter alia, be carried out via karyotyping, analysis of DNA-fragmentation and/or a spermiogram for example all of which are considered standard/routine methods in the art. A “higher risk” of leading to congenital abnormality or aneuploidy may be defined, for example, by elevated levels of DNA fragmentation, elevated oxidation-reduction potential, elevated ROS levels, or a higher fraction of gametes with an unusual number of chromosomes when the patients' gametes are compared to the gametes of a normal control patient which were treated the same way as the patients' gametes. Since haploid gametes contain 23 chromosomes, any number of chromosomes different from 23 may represent an unusual number of chromosomes in the assessed gametes. As already detailed herein above, for example, the Male Infertility Oxidative System (MiOXSYS, Englewood, CO) may be used to determine (elevated) ROS levels and/or (elevated) oxidation-reduction potential of spermatozoa. The term “elevated” refers to (ROS) levels that can be (clearly) distinguished from average or median (ROS) levels of a control group or from a control sample. As detailed herein below, appended FIGS. 7D and 8D show DNA-fragmentation and oxidation-reduction potential measurements of sperm samples that were not treated with carbon monoxide from individual subjects. Here, only few sperm samples show high measurements regarding oxidation-reduction potential, while the majority of samples show comparable (low) values for oxidation-reduction potential. It is conceivable that the sperm samples with low oxidation-reduction potential may be considered control samples.

Once it may be established that a patient or a patients' gametes are in need of CO-treatment, the patients' gametes may be collected and subjected to the method as described herein above. As already described herein above, the patients' gametes may also be contacted and/or treated with CO in vivo. Here, CO may be administered systemically and/or transdermally to said gametes. Systemic administration may include but is not limited to the administration of a CO-releasing system/a CO-releasing suppository via including but not limited to oral, rectal, and/or intraurethral administration. Transdermal and/or transcutaneous administration of CO may include but is not limited to application of CO or a CO-releasing system to the patients' scrotum. The CO gas may diffuse through, inter alia, epithelial layers and contact said gametes. Here, the CO-releasing system may be attached and/or glued to the patients' scrotum. In one aspect, said CO-releasing system may be a patch. CO-releasing patches are well known in the art and have been described in WO 2021/180908 A1 and Ruopp et al. (2023, Journal of Controlled Release), which are herewith incorporated by reference in their entirety. Such patches may be used in the treatment of wounds, inflammatory diseases of the skin, and inflammatory diseases of subcutaneous skin tissue, joints, and tendons, however, their use in treating gamete diseases has not been disclosed before. To allow for sufficient treatment of gametes it order to obtain the desired effects of CO in vivo, said gametes may be contacted with CO (for example using the above-mentioned CO-releasing systems/patches) for at least 240 min, at least 300 min, at least 360 min before collection of the gametes. In one aspect, the in vivo treatment of said gametes may be conducted multiple times, namely at least 1 time, at least 2 times, at least 3 times, at least 4 times, at least 5 times, at least 6 times with CO each time for at least 240 min, at least 300 min, at least 360 min before the collection of the gametes. The scrotum may be contacted with between about 90 μmol and about 540 μmol carbon monoxide. Evidently, the in vivo treatment of gametes with CO is not mutually exclusive with the ex vivo treatment of gametes with CO. In other words, gametes may be first contacted with CO in vivo using the means and methods disclosed herein and may after collection of the gametes be contacted with CO ex vivo using the means and methods disclosed herein.

In vivo CO administration to a patients' gametes may reduce oxidation-reduction potential/ROS-levels and/or DNA-fragmentation of the gametes and consequently improve their viability. As already described in detail herein above, ROS levels can increase and/or accumulate in gametes of an individual due to various factors, including but not limited to stress. Namely, ROS levels in an individual may increase through, inter alia, (physiological and/or psychological) stress, genetic predisposition, environmental factors (including exposition to electromagnetic waves), and/or behavioral risk factors (diet, including smoking, alcohol consumption, and/or drug abuse). Systemic ROS levels may be linked to the ROS levels of gametes and may, thus, be used as an indication of the ROS levels of an individuals' gametes. Systemic ROS levels may be assessed by assessing ROS levels in an individuals' urine and/or blood sample, in particular in a serum sample. Means and methods to determine, inter alia, ROS levels in an individuals' serum sample are well known in the art and may be conducted using the RedoxSys device from Aytu Biosciences (Aytu Biosciences, Inc, Englewood, CO 80112, USA).

ROS in gametes can affect reproductive efficiency of artificial reproductive technologies and natural fertilisation during, inter alia, copulation. Thus, it is evident that gametes that were contacted with CO in vivo and consequently show reduced ROS-levels are particularly suited for subsequent artificial reproduction techniques and/or subsequent natural fertilization and may improve the success and/or success-rates thereof. As already described herein above, a reduction of ROS-levels by (in vivo) CO treatment may prevent congenital abnormality and/or aneuploidy, treat and/or reduce male infertility, treat and/or prevent a disease caused by and/or linked to elevated DNA fragmentation and/or oxidation-reduction potential of gametes, or treat and/or prevent a disease of gametes caused by/linked to elevated ROS levels.

In another embodiment, the present invention relates to a kit comprising the CORM and the second compound in a closed compartment (“CORS”) as described in detail herein above. Preferably, such a kit may further comprise instructions describing the method and/or the use as described herein above. Such a kit may further also contain additional components such as the container, buffers etc. which may be used/useful in the method as described above. Such a kit may preferably be a home kit which can be used by everybody who can perform the method as described above or a kit that may be used by specifically trained staff in e.g., a fertilization clinic for example.

Further embodiments are exemplified in the scientific part. The appended figures provide illustrations of the present invention. Whereas the experimental data in the examples and as illustrated in the appended figures are not considered to be limiting, the technical information comprised therein forms part of this invention. The invention thus also covers all further features shown in the figures individually, although they may not have been described in the previous or following description. Also, single alternatives of the embodiments described in the figures and the description and single alternatives of features thereof can be disclaimed from the subject matter of the other aspect of the invention.

FIG. 1A: Illustrative workflow of the method of the present invention for the preservation of spermatozoa using carbon monoxide.

FIG. 1B: Illustrative workflow of the method of the present invention for the preservation of spermatozoa using carbon monoxide. The workflow illustrates the process of sample collection and CO treatment (using a CO-releasing system; CORS) for the options of home use and use in a professional area, such as an ART laboratory.

FIG. 2: Results of sORP measurements on sperm samples from 6 subjects. Shown are the relative sORPs of the fraction of sperms treated with CO (wCO) as compared to the fraction of sperm without CO (woCO, set to 1).

FIG. 3: Results from DNA fragmentation measurements on sperm samples from 3 subjects. Shown are relative rates of DNA fragmentation of the fraction of sperms treated with CO (wCO) as compared to the fraction of sperm without CO (woCO, set to 1).

FIG. 4: Results from sperm motility measurements on sperm samples from 5 subjects. A: total mobility, B: Progressive motility. Shown are relative values of the fraction of sperms treated with CO (wCO) as compared to the fraction of sperms without CO (woCO, set to 1).

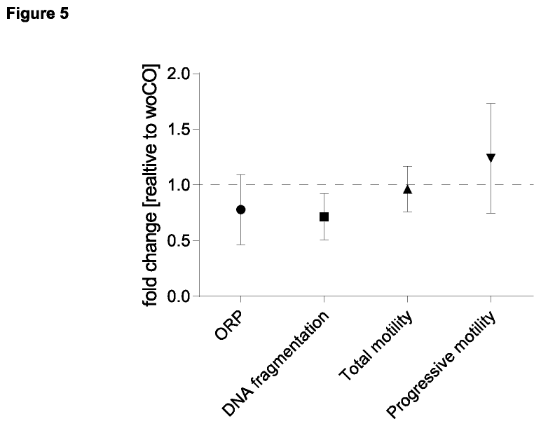

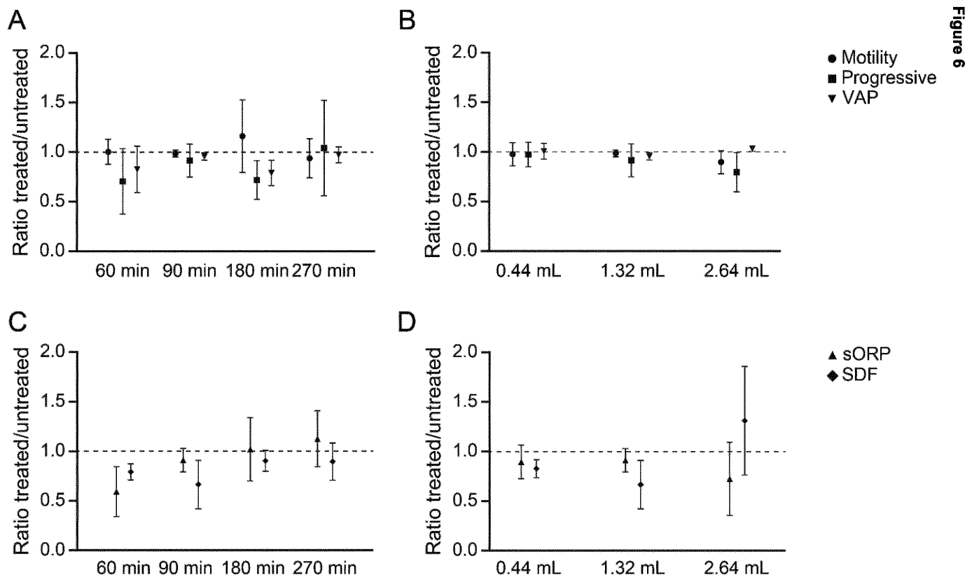

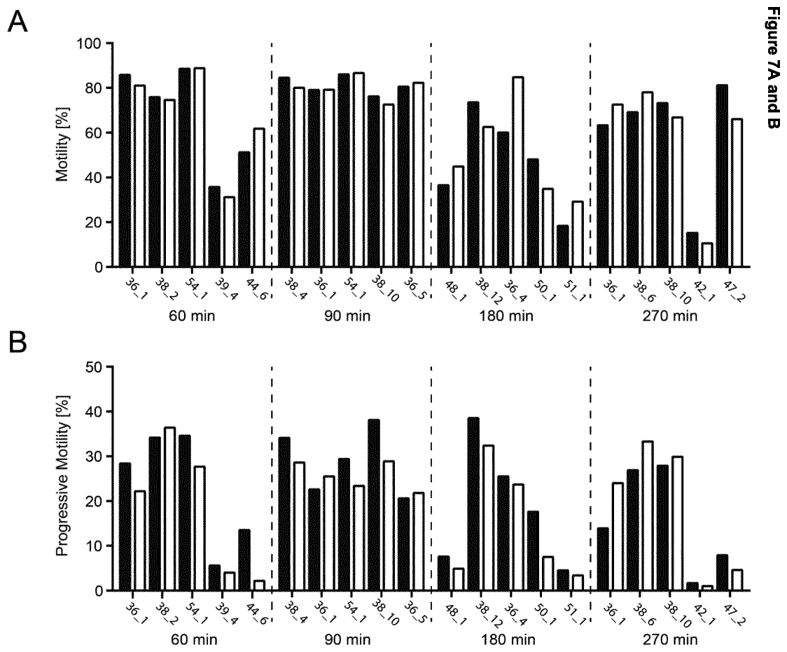

FIG. 5: Overview of relative changes of parameters of sperm with CO (wCO) compared to sperm without CO (woCO; set to 1). Data are represented as mean±SD; n=5 (total and progressive motility, see FIG. 4), n=6 (sORP, see FIG. 2), n=3 (DNA fragmentation, see FIG. 3).

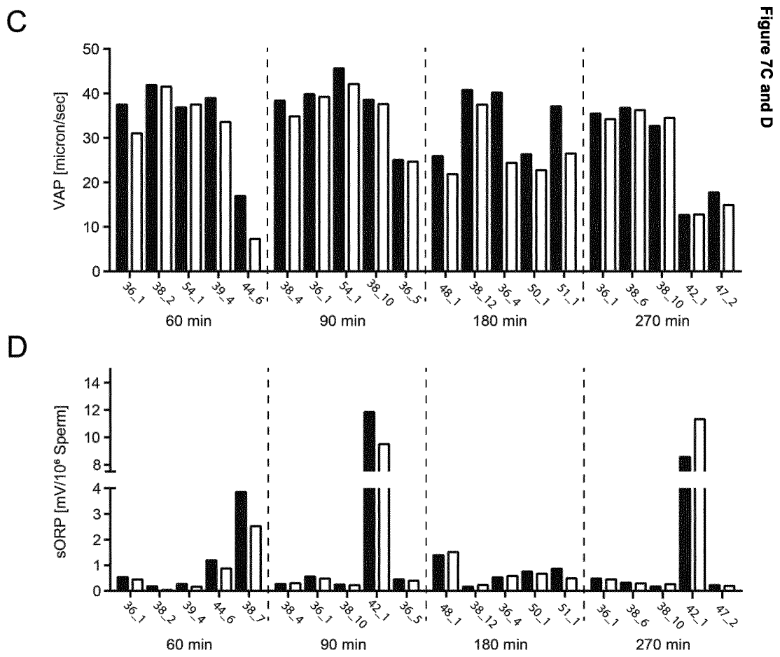

FIG. 6: Relative changes in sperm viability and motility parameters in response to different CO regimens. A Ratio treated/untreated of motility (“Motility”), progressive motility (“Progressive”), and VAP (“VAP”) with a dose of 1.32 mL CO and different exposure times; B Ratio treated/untreated of motility, progressive motility, and VAP with different doses of CO at 90 minutes exposure time; C Ratio treated/untreated of oxidation-reduction potential (“sORP”) and DNA-fragmentation (“SDF”) to the control with a dose of 1.32 mL CO and different exposure times; D Ratio treated/untreated of oxidation-reduction potential and DNA-fragmentation with different doses of CO at 90 minutes exposure time; values are presented as mean±SD, n=5.

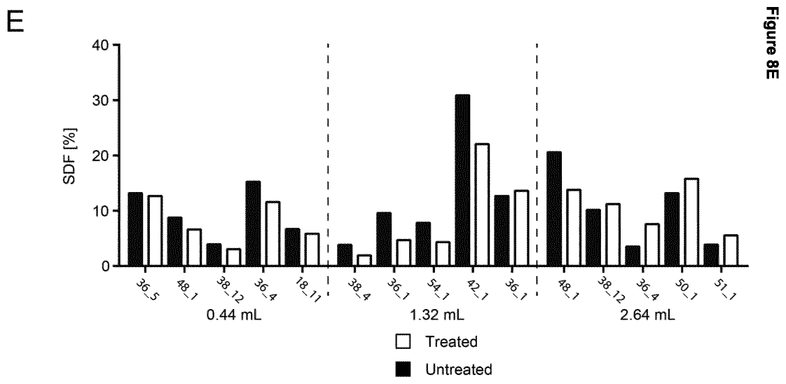

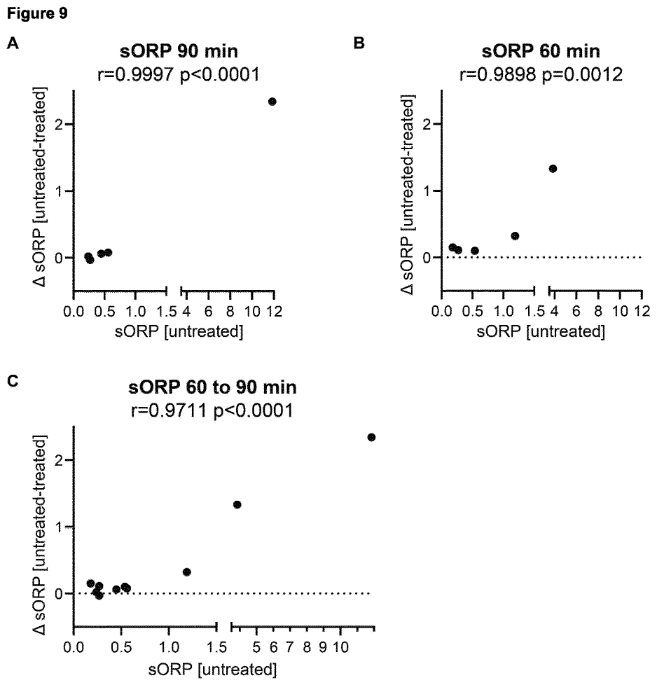

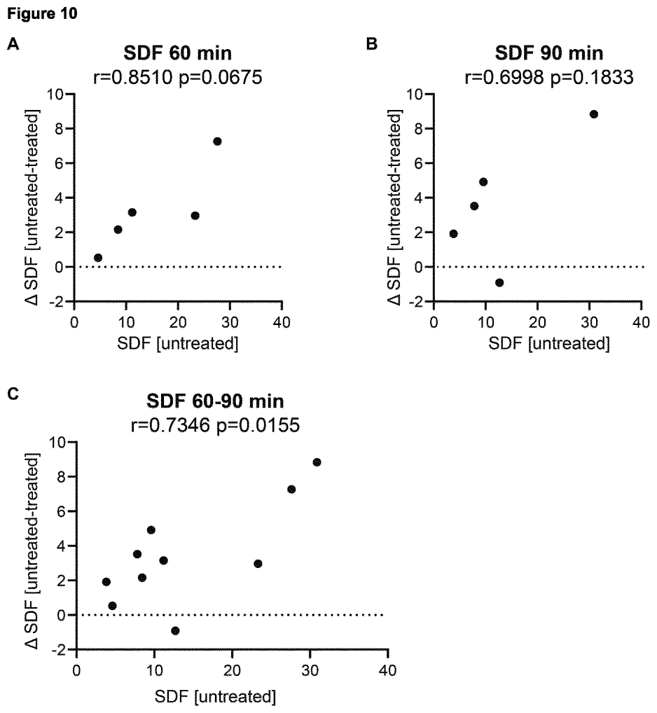

FIG. 7: Paired measurements from individual subjects with and without CO treatment at different incubation periods. The ejaculate of each subject was collected in a sample cup, divided into equal parts, and treated with and without CORS (1.32 mL CO gas) for different time periods (60-270 min). After the different periods, motility (A), progressive motility (B), and mean velocity (VAP) (C) were analyzed by computerized sperm analysis. Sperm DNA fragmentation (SDF) (E) was determined by microscopy and static oxidation-reduction potential (SORP) (D) by the MiOXSYS® system. Numbers under the x-axis indicate subject-IDs. The data of FIG. 7 is summarized in FIG. 6 and partially summarized in FIGS. 9 and 10.

FIG. 8: Pairwise measurements of individual subjects with and without CO treatment at different concentrations. Each subject's ejaculate was collected in a sample cup, divided into equal parts, and treated with and without CORS (0.44, 1.32, or 2.64 mL CO gas) at different concentrations for 90 minutes. After 90 minutes of treatment, motility (A), progressive motility (B), and average velocity profile (VAP) (C) were analyzed by computerized semen analysis. Sperm DNA fragmentation (SDF) (E) was determined by microscopy and static oxidation-reduction potential (sORP) (D) by the MiOXSYS® system. Numbers under the x-axis indicate subject-IDs. The data of FIG. 8 is summarized in FIG. 6.

FIG. 9: Linear correlation between sORP levels in untreated sperm samples and the effectiveness of CO treatment on sORP levels of said sperm samples. Assessment of the correlation between static oxidation-reduction potential (sORP) values in the non-treated group and the AsORP values (difference between untreated and treated) following 60 min (A) (according to WHO guidelines) or 90 min (B) (max duration within the ART laboratory) of incubation with CORS (15 mg CORM), as well as encompassing the entire 90-min period (C). r: Coefficient of correlation; p: significance level. Measurements on the x- and y-axes are in [mV/106 sperm].

FIG. 10: Linear correlation between SDF levels in untreated sperm samples and the effectiveness of CO treatment on SDF levels of said sperm samples. Assessment of the correlation between Sperm DNA fragmentation (SDF) values in the non-treated group and the ASDF values (difference between untreated and treated) following 60 min (A) (according to WHO guidelines) or 90 min (B) (max duration within the ART laboratory) of incubation with CORS (15 mg CORM), as well as encompassing the entire 90-min period (C). r: Coefficient of correlation; p: significance level. Measurements on the x- and y-axes are in [%].

FIG. 11: A schematic illustration of a side view of an exemplary treatment system according to an embodiment of the present invention. The highlighted (technical) features are described in detail herein above.

FIG. 12: A schematic illustration of a side view of an exemplary treatment system according to a further embodiment of the present invention. The highlighted (technical) features are described in detail herein above.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The methods and techniques of the present invention are generally performed according to conventional methods well known in the art and as described in various general and more specific references that are cited and discussed throughout the present specification unless otherwise indicated. See, e.g., WHO laboratory manual for the examination and processing of human semen, 6^{th} edition; WHO 2021; Madigan et al., Brock Biology of Microorganisms, 15^{th} ed., Pearson (2018); Berg et al., Stryer Biochemie, 7^{th} ed., Springer Spektrum (2013).

While the invention has been illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

The invention also covers all further features shown in the figures individually, although they may not have been described in the afore or following description. Also, single alternatives of the embodiments described in the figures and the description and single alternatives of features thereof can be disclaimed from the subject matter of the other aspect of the invention.

Furthermore, in the claims, the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

In this specification, a number of documents including scientific publications, patent applications, and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

In accordance with the above, the present invention relates to, inter alia, the following items, whereby further embodiments of the present invention have been explained above and are also illustrated in the appended examples and figures:
1. Method for the preservation of gametes, the method comprising the steps of
   a) providing/obtaining gametes or a sample comprising said gametes in a container and
   b) contacting said gametes or said sample comprising said gametes in said container with carbon monoxide.
2. The method according to item 1, wherein said gametes or said sample comprising said gametes comprise spermatozoa or egg cells, preferably wherein said spermatozoa are comprised within seminal fluid, ejaculate and/or a buffer/buffer system.
3. The method according to items 1 and 2, wherein said carbon monoxide is released from a carbon monoxide releasing molecule and/or wherein said carbon monoxide is directly supplied to said container via a separate tube.
4. The method according to any one of items 1 to 3, wherein said carbon monoxide forms a gas layer over said gametes or over said sample comprising said gametes.
5. The method according to items 3 and 4, wherein said carbon monoxide releasing molecule is preferably a metal carbonyl compound, even more preferably a molybdenum carbonyl compound, most preferably Na₃Mo(CO)₃(CNCH₂CO₂H)₃.
6. The method according to any one of items 3 to 5, wherein said carbon monoxide releasing molecule is to be contacted with FeCl₃, Ce(SO₄)₂ or H₂O₂, preferably with FeCl₃ thereby releasing said carbon monoxide.
7. The method according to any one of items 1 to 6, wherein said gametes or said sample comprising said gametes are contacted with said carbon monoxide for about 15 to about 120 minutes, preferably for about 30 to about 90 minutes, more preferably for about 30 to about 60 minutes.
8. The method according to any one of items 1 to 7, wherein said gametes or said sample comprising said gametes are contacted with said carbon monoxide at about 25 °C to about 35 °C.
9. The method according to any one of items 1 to 8, wherein the amount of carbon monoxide applied to said gametes or said sample comprising said gametes is between about 20 μmol and about 500 μmol, about 40 μmol and about 400 μmol, preferably between about 45 μmol and about 270 μmol, preferably between about 60 μmol and about 180 μmol.
10. The method according to any one of items 1 to 9, wherein said gametes are human gametes.
11. The method according to any one of items 1 to 9, wherein said gametes are gametes from cattle, horses, pigs, sheep, goats, camels, alpacas, dogs, cats, birds, or fish.
12. The method according to any one of items 1 to 11, wherein the preservation of said gametes is assessed via determination of motility, DNA-fragmentation, spermiogram and/or oxidation-reduction potential of said gametes.
13. A method for reducing DNA-fragmentation and/or oxidation-reduction potential of gametes in an artificial reproductive technology, wherein said gametes are or a sample comprising said gametes is contacted with carbon monoxide.
14. Use of carbon monoxide for reducing DNA-fragmentation and/or oxidation-reduction potential of gametes in an assisted reproductive technology, wherein said gametes are or a sample comprising said gametes is contacted with carbon monoxide.
15. The method according to item 13, or the use according to item 14, wherein said assisted reproductive technology comprises in vitro fertilization, intracytoplasmic sperm injection, intrauterine insemination, frozen embryo replacement, preimplantation genetic testing, in vitro maturation of oocytes, frozen oocyte replacement, gamete intrafallopian transfer, zygote intrafallopian transfer, and/or cryopreservation.
16. The method according to item 13 or 15, or the use according to item 14 or 15, wherein reducing DNA-fragmentation and/or oxidation-reduction potential of gametes maintains/increases motility of said gametes.
17. The method according to any one of items 13, 15, and 16, or the use according to any one of items 14 to 16, wherein reducing DNA-fragmentation and/or oxidation-reduction potential of gametes reduces the risk of congenital abnormality and/or aneuploidy in an artificial reproductive technology.
18. Carbon monoxide for use in preventing congenital abnormality and/or aneuploidy.
19. Carbon monoxide for use in treating/reducing male infertility.
20. Carbon monoxide for use in treating and/or preventing a disease caused by/linked to elevated DNA fragmentation and/or oxidation-reduction potential of gametes.
21. Carbon monoxide for use in treating and/or preventing a disease of gametes caused by/linked to elevated ROS levels.
22. The carbon monoxide for use according to item 20 or 21, wherein said disease is caused by/linked to elevated stress levels or wherein the individual to be treated suffers from stress.
23. The carbon monoxide for use according to item 22, wherein said disease is caused by/linked to elevated psychological and/or physiological stress levels or wherein the individual to be treated suffers from psychological and/or physiological stress.
24. The carbon monoxide for use according to any one of items 18 to 23, wherein preventing congenital abnormality and/or aneuploidy, treating/reducing male infertility, or treating/preventing said disease is characterized by contacting gametes with carbon monoxide.
25. The carbon monoxide for use according to item 24, wherein said gametes are contacted with carbon monoxide in vitrolex corpore.
26. The method according to any one of items 13, and 15 to 17, the use according to any one of items 14 to 17, or the carbon monoxide for use of item 25, wherein said gametes are or said sample comprising said gametes is contacted with said carbon monoxide less than 10 min after collection of said gametes in said container, preferably less than 5 min, more preferably less than 3 min, more preferably less than 2 min, more preferably less than 1 min.
27. The carbon monoxide for use according to item 24, wherein said gametes are or said sample comprising said gametes is contacted with carbon monoxide in vivo.
28. The carbon monoxide for use according to item 27, wherein said carbon monoxide is administered systemically or transdermally to said gametes.
29. The method according to item 17, the use according to item 17, or the carbon monoxide for use according to any one of items 17, and 24 to 28, wherein said congenital abnormality is selected from congenital abnormality of the limb, congenital abnormality of the heart, congenital abnormality of the nervous system, congenital abnormality of the gastrointestinal system, and congenital abnormality of the lung.
30. The method according to item 29, the use according to item 29, or the carbon monoxide for use according to item 29, wherein said congenital abnormality is congenital abnormality of the limb selected from the group consisting of achondroplasia, amelia, amniotic band syndrome, brachydactyly, cleidocranial dysostosis, congenital aplasia or hypoplasia, ectrodactyly, oligodactyly, phocomelia, polydactyly, polymelia, polysyndactyly, and syndactyly.
31. The method according to item 29, the use according to item 29, or the carbon monoxide for use according to item 29, wherein said congenital abnormality is congenital abnormality of the heart selected from the group consisting of atrial septal defect, patent ductus arteriosus, tetralogy of fallot and ventricular septal defect.
32. The method according to item 29, the use according to item 29, or the carbon monoxide for use according to item 29, wherein said congenital abnormality is congenital abnormality of the nervous system selected from the group consisting of neural tube defects such as agenesis of the corpus allosum, anencephaly, Arnold-Chiari malformation, Dandy-Walker malformation, encephalocele, holoprosencephaly, hydrocephalus, lissencephaly, megencephaly, meningocele, meningomyelocele, microencephaly, polymicrogyria, and spina bifida.
33. The method according to item 29, the use according to item 29, or the carbon monoxide for use according to item 29, wherein said congenital abnormality is congenital abnormality of the gastrointestinal system selected from the group consisting of atresia, imperforate and stenosis.
34. The method according to item 29, the use according to item 29, or the carbon monoxide for use according to item 29, wherein said congenital abnormality is congenital abnormality of the lung, preferably wherein said congenital abnormality of the lung is congenital bronchiectasis.

### Example 1

This example outlines an illustrative workflow of the method of the present invention for the preservation of spermatozoa using carbon monoxide. This exemplary workflow is also illustrated in appended FIG. 1.

In a first step, the carbon monoxide (CO) releasing system (CORS) comprising (i) the carbon monoxide releasing molecule (CORM, like Na₃Mo(CO)₃(CNCH₂CO₂H)₃; Mo-CORM) and (ii) a second compound such as FeCl₃, Ce(SO₄)₂ or H₂O₂, preferably FeCl₃ is provided in a closed compartment (such as a capsule) which is permeable for CO gas. (Mo-)CORM and the second compound are at this point not in contact with each other within the closed compartment. In the next step, the spermatozoa are harvested by/from a healthy or diseased subject/patient and transferred into a container. Simultaneously, the (Mo-)CORM is contacted with a second compound like an aqueous solution of FeCl₃, Ce(SO₄)₂ or H₂O₂, within the closed compartment/capsule (“activated CORS”), by applying force to both ends of the longitudinal axis of the capsule. This releases carbon monoxide gas from the (closed) compartment/capsule. The closed compartment which releases carbon monoxide gas is then added to the container containing the spermatozoa sample. The container is closed, and the samples are incubated for a given time (preferably 25-35 °C for 30 to 60 minutes) and transferred to an assisted reproductive technologies (ART) laboratory. During incubation liquefication takes place naturally at the given conditions. Liquefaction in this context describes the process of braking up the gel formed by proteins from the seminal vesicles and the prostate in order for the semen/ejaculate to become more liquid. Finally, the CORS (=the closed compartment containing the CORM such as Mo-CORM, and FeCl₃, Ce(SO₄)₂ or H₂O₂, preferably FeCl₃) is removed from the container and the sample is further processed for ART or is cryopreserved.

Dosing (number of added CORS/amount of (Mo-)CORM) is dependent on the load of the system with Mo-CORM and the resulting maximal amount of CO which can be released. 1 mg of Mo-CORM can release a maximum of 6 μmol of CO gas. The Mo-CORM load of the CORS depends on the size of the system. Detailed specifications of size and load of Mo-CORM and FeCl₃ are described in PCT/EP2020/078794, which is published as WO 2021/074159 A1. Further specification can be found in EP22216317.2, filed on Dec. 23, 2022, and in PCT/EP2023/074808, filed on Sep. 8, 2023.

### Example 2

Example 2 illustrates the improvement of characteristics of spermatozoa (like oxidation-reduction-potential, DNA fragmentation and/or spermatozoa motility, in particular progressive motility) when contacted with carbon monoxide gas as compared to “untreated” control spermatozoa from the same patient(s) which were not contacted to carbon monoxide gas. The results of this example are shown in appended FIGS. 2 to 5.

### Methods

In this example, one CORS-capsule (18-22 mm in length and 6-8 mm diameter) loaded with 15 mg of Mo-CORM and 150 μl of an aqueous FeCl₃ solution (583.3 mg/ml) was used for the preservation of a 0.7 mL to about 2.9 mL sample in a standard 15 mL Falcon tube.

CORS, Mo-CORM and FeCl₃ were prepared as described below:

### Preparation of CORS

The CORS was produced by additive manufacturing. The two compartments separated by a septum contained in one of the compartments were 3D printed. The compartment containing the aqueous solution of FeCl₃ was printed drop-on-demand (poly-jet modelling process) using an Objet Eden 350 printer (96 nozzles, drop size 40 μM, resolution X=600 dpi, Y=600 dpi; Stratasys, Deden Prairie MN, USA). As resin, the photopolymer VeroBlackPlus (Stratasys, Deden Prairie, MN) was used, and the structure was cured via UV-polymerization at 365 nm using a UV lamp. Furthermore, a water-soluble support structure (SUP705, Stratasys, Deden Prairie, MN) was used, which was removed after the printing process by washing steps (potable water; isopropanol).

The compartment containing Mo-CORM was produced by laser sintering (LS) using polyamide PA2200 (EOS GmbH, Krailing, Germany) as material, processed by a Formiga P110 3D printer (EOS GmbH, Krailling, Germany), including a CO₂-laser (wavelength 10.6 μm, layer thickness 0.1 mm). The silicone membrane (SIK8649), which surrounds the two compartments, was obtained from RAUMEDIC AG (Helmbrechts, Germany). A CAD software (VISI 2019 & 2020, Vero UK, Cheltenham, UK) was used for the production-ready design.

### Preparation of Mo-CORM

The synthesis of the CORM trisodiumtricarbonyl-[tris(isocyanoethylacetate)]molybdenum (Na₃Mo(CO)₃(CNCH₂CO₂)₃, Mo-CORM) was modified from previous protocols (Achatz, D et al. Zeitschrift für anorganische und allgemeine Chemie 2005, 631 (12), 2339-2346). In brief, under dry and inert conditions, 2.64 g (10.0 mmol) of molybdenum hexacarbonyl was dissolved in 35 mL anhydrous acetonitrile (99.8%; 90 °C; 22 h) to yield the intermediate acetonitrile complex. After that, 4.7 mL ethylisocyanoacetate (3.5 eq.; 35 mmol; 4.86 g) was added to replace the acetonitrile at 55 °C to obtain complex. Next, the ester was hydrolyzed using NaOH (16 eq.) in 20 mL tetrahydrofuran at room temperature. By adding aqueous HCl, the free acid is formed. Finally, ethanolic NaOH (5 mmol NaOH/1 mmol Mo-CORM) was added to obtain the trisodium-salt. All reagents were purchased from Sigma Aldrich (Schnelldorf, Germany) and used without further purification.

### Preparation of Aqueous FeCl₃ Solution

FeCl₃*6H₂O was purchased from Sigma Aldrich (Schnelldort, Germany). An aqueous solution was prepared by preparing a concentration of 583.3 mg/mL with deionized water.

### Sperm Analysis

Between 1.46 mL and 5.7 mL human semen/spermatozoa were collected from healthy males of between 25 and 50 years of age in 100 mL sample vials on site. Samples were then divided into two equal fractions each for subsequent incubation with CO (wCO) and without CO (woCO). In order to estimate the spermatozoa concentration in the samples and to establish an initial baseline for each sample, a routine spermiogram was performed before samples were transferred to gas-tight 15 mL Falcon tubes. Samples were incubated at 25-35 °C for 90 minutes with and without an activated CORS system in a closed container. During this incubation period liquefaction took place naturally. The samples were then analyzed for total motility, progressive motility, static oxidation-reduction potential, and for DNA fragmentation. Total motility, progressive motility and concentration of the samples were assessed using a CEROS II (Hamilton Thorne, Beverly, MA) computer assisted sperm analysis (CASA) system. 6 μL of a sperm sample diluted 1:1 with Multipurpose Handling Medium-Complete (MHM-C, FujiFilm/IrvineScientific, Santa Ana, CA) was measured and the concentration and level of mobility (total and progressive motility) was assessed.

A Male Infertility Oxidative System (MiOXSYS, Englewood, CO) was used to determine static oxidation-reduction potential (sORP) levels. 30 μL of the sperm samples were transferred to the sample application port on a MiOXSYS sensor and analyzed for 2 minutes.

To determine DNA fragmentation a count-based microscopy assay was performed. For this spermatozoa samples were diluted using MHM-C (Multipurpose Handling Medium-Complete, FujiFilm, IrvineScientific) to a maximum of 20×E6 spermatozoa per mL and analyzed using a Halo Sperm G2 Kit (Halotech, Madrid, Spain). Samples were mixed with the prepared agarose according to manufacturer instructions. 8 μL of the resulting mixture was then put on microscopy slides and cooled down for 5 minutes by removing the heat source. The prepared samples were further treated according to the manufacturer's instructions: denaturant for 7 minutes, lysis solution for 20 minutes, distilled water for 5 minutes, 70% ethanol for 2 minutes, 100% ethanol for 2 minutes, eosin staining solution for 7-10 minutes, and thiazine staining solution for 7-10 minutes. At least 300 spermatozoa were then analyzed using a bright field microscope.

### Results

1. (Static) Oxidation-Reduction Potential (sORP) Levels

“Relative oxidation-reduction-potential” data points in FIG. 2 are derived from the following fraction per test subject: sORP wCO_{Subject n}/SORP woCO_{Subject n}, wherein sORP woCO_{Subject n} was set to 1. sORP data as a measure for oxidative stress (caused among others by ROS) of the sperms/spermatozoa show a trend to be lower in the samples that were contacted with CO (wCO) as compared to samples that were not contacted with CO (woCO). This is evident from the location of the data points, four of which are located below the dashed woCO=1 reference line. Therefore, without being bound by theory, these results indicate that the contacting with CO leads to lower levels of oxidative oxygen species (ROS) (FIG. 2) in spermatozoa as compared to spermatozoa that were not contacted with CO.

### 2. DNA Fragmentation

“Relative DNA fragmentation” data points presented in FIG. 3 are derived from the following fraction per subject: DNA fragmentation wCO_{Subject n}/DNA fragmentation woCO_{Subject n}, whereas DNA fragmentation woCO_{Subject n} was set to 1. As is evident from FIG. 3, DNA fragmentation is reduced in samples that were contacted with CO as compared to samples that were not contacted with CO. This is evident from the location of the data points which are all located below the dashed woCO=1 reference line.

### 3. Spermatozoa Motility (Total Motility and Progressive Motility)

“Relative total motility” or “relative progressive motility” data points presented in FIGS. 4A and B respectively, are derived from the following fractions per subject:
a) for relative total motility: total motility wCO_{Subject n}/total motility woCO_{Subject n}
b) for relative progressive motility: progressive motility wCO_{Subject n}/progressive motility woCO_{Subject n},
wherein total motility/progressive woCO was set to 1 respectively. While no differences between samples that were contacted with CO (wCO) and without CO (woCO) can be observed for total motility (most data points are located close to the dashed woCO=1 reference line, see FIG. 4A) the progressive motility is increased for wCO as compared to woCO (as is evident from the fact that with exception of subject 6, all data points are located on or above the dashed woCO=1 reference line, see FIG. 4 B).

In this context, total motility refers to the percentage of sperm making any sort of movement. This movement can include non-progressive movement. Progressive motility refers to sperm that are swimming in a mostly straight line or large circles and may, therefore, be an important predictive parameter for the success of an ART cycle.

There, progressive motility is increased in spermatozoa that were incubated with CO as compared to spermatozoa that were not incubated with CO.

In summary, the results show that the contacting of spermatozoa with CO leads to desired/improved characteristics of gametes (like to a reduction in oxidative stress/oxidation-reduction potential/ROS levels (FIG. 2) and DNA fragmentation (FIG. 3) as well as to an improvement of their progressive motility (FIG. 4 B)). All of the measured parameters are again comprehensively illustrated in FIG. 5. FIG. 5 indicates the mean of the data points as presented in FIGS. 2 to 4 for each measured parameter/characteristic, wherein the respective reference level for samples that were not contacted with CO (woCO) is again set to 1 (dashed line). As is evident from FIG. 5, contacting of spermatozoa with CO leads to (i) a reduction in oxidative stress/ROS (“ORP”; mean below the dashed reference line indicates that the mean wCO value is smaller than the mean woCO value), (ii) a reduction in DNA fragmentation (mean below the dashed reference line indicates that the mean wCO value is smaller than the mean woCO value) as well as to (iii) an improvement of progressive motility (mean above the dashed reference line indicates that the mean wCO value exceeds the mean woCO value).

Therefore, carbon monoxide (CO) when contacted to gametes like spermatozoa was surprisingly found by the inventors to lead to preservation of spermatozoa. As a consequence of the co-incubation of gametes with CO, these spermatozoa have been shown to exhibit desired characteristics (compared to spermatozoa that were not contacted with CO) and are thus particularly useful for ART (applications).

### Example 3

Example 3 illustrates the improvement of spermatozoan viability parameters (like the reduction of DNA fragmentation and oxidation-reduction potential) in response to different CO/Mo-CORM regimens (CO inculation duration and CO/Mo-CORM load). Further, this example illustrates the maintenance of spermatozoan motility (like total motility, progressive motility, and average path velocity (VAP)) in response to different CO/Mo-CORM regimens (CO incubation duration and CO/Mo-CORM load). Sperm form the same subject was either treated with CO gas or left untreated, as “untreated” control. Treatments with CO varied in duration (60, 90, 180, or 270 min) and in CO/Mo-CORM load (0.44, 1.32, or 2.64 mL of CO, corresponding to 5, 15, or 30 mg of Mo-CORM). The results from this example are shown in appended FIGS. 6 to 10.

FIG. 6 depicts the ratio of the respective measurements of CO-treated and untreated sperm samples obtained from the same subject. Here, no effect of CO-treatment would correspond of a value of 1, as indicated by the dotted horizontal line. Any value above the dotted line corresponds to an increment in the respective measurement by CO-treatment as compared to the untreated control, whereas any value below the dotted line corresponds to a reduction in the respective measurement by CO-treatment as compared to the untreated control. FIGS. 7 and 8 illustrate the single measurements that are summarized in FIG. 6. In FIGS. 7 and 8, each bar corresponds to a single measurement, wherein neighboring white and black bars correspond to paired CO-treated and untreated sperm samples obtained from the subject, respectively. Thereby, each dot in FIG. 6 corresponds to the ratio of a paired CO-treated and untreated sample shown in FIGS. 7 and 8. Thus, FIGS. 7 and 8 clearly confirm the observations made on the basis of FIG. 6. Panels D and E of FIGS. 7 and 8 depict the oxidation-reduction potential and DNA fragmentation measurements of sperm samples from individual subjects, respectively. Here, high variation between untreated sperm samples from different subjects can be observed for both oxidation-reduction potential and DNA fragmentation, clearly demonstrating that among the tested subjects the sperm of several subjects suffers from extraordinarily high oxidative stress when left untreated. FIGS. 7 and 8 further clearly demonstrate that CO treatment is able to reduce oxidation-reduction potential and DNA fragmentation in such subjects. This is in particular true for sperm samples treated for 60 or 90 minutes. FIGS. 9 and 10 depict correlative analyses of the effect of the CO treatment (difference of treated and untreated samples) with the respective measurements of the untreated sperm samples. FIGS. 9 and 10 demonstrate that CO treatment is particularly effective in subjects that suffer from high sperm oxidation-reduction potential and/or DNA-fragmentation.

### Methods

In this example, sperm samples were treated with either one, three, or six CORS capsules, each loaded with 5 mg Mo-CORM (corresponds to 0.44 mL CO) for 60 to 270 mins, or left untreated for the same amount of time.

CORS and Mo-CORM were prepared using the materials and as described below. FeCl₃ (prepared as described in Example 2) was used for Mo-CORM activation.

### Materials

All chemicals needed for the synthesis of Mo-CORM as well as activation including molybdenum hexacarbonyl, ethylisocyanoacetate, acetonitrile, anhydrous tetrahydrofuran, sodium hydroxide p.a., hydrochloric acid p.a., absolute ethanol, FeCl₃*6H₂O, nitric acid (65%, p.a.) were purchased from Sigma-Aldrich Chemie GmbH (Schnelldorf, Germany).

Multipurpose Handling Medium (MHM) for sperm dilution was purchased from IrvineScientific (Santa Ana, CA). 270 ppm CO calibration gas was purchased from Linde AG (Munich, Germany). The polyamide (PA2200) and duroplast photopolymer MED610+VeroBlackPlus (RGD875) were supplied by EOS GmbH (Krailling, Germany) and Stratasys Ltd. (Rechovot, Israel), respectively. Silicone R 6.65×0.4 mm was purchased from RAUMEDIC AG (Helmbrechts, Germany). SF33 2K-silicone (Mixing ratio 1:1, viscosity before mixing: 7000-8000 cP, stiffness after mixing: 33 ShA, density 1.11 g/cc, breaking point: 4.7 N/mm²) and SF45 2K-silicone (Mixing ratio 1:1, viscosity before mixing: 8500 cP (at 23 °C), stiffness after mixing: 45 ShA, density: 1.12 g/cc, breaking point: 3.5±0.5 N/mm²) were purchased from Silikonfabrik (Ahrensburg, Germany). All casting molds were constructed from polytetrafluorethylene (Vink, Germany) on a CNC machine type neo (Datron AG, Germany). Loctite SI 5248, Loctite 4902, Loctite HY 4011, and Loctite SF7701 were purchased from Henkel (Düsseldorf, Germany). Sterican® needles 0.9*40 mm and Omnican® U100 Insulin needles 0.3*8 mm were purchased from B. Braun (Melsungen, Germany). All other reagents were purchased from Sigma Aldrich Chemie GmbH and at least of pharmaceutical grade unless otherwise noted.

### Manufacturing of CORS

All parts were molded using custom designed Teflon casting molds. After filling the molds, vacuum was applied for 5 minutes to extract any air bubbles. The cast was then filled again to the rim and the vacuum was applied for another 5 minutes. After that time the corresponding cast was pressed into the silicone and fixed with clamps. After drying at 40 °C for 24 hours the casted molds were separated from the cast with pressured air. The vessel and lid were casted separately. The vessel was filled with Mo-CORM. The lid and upper rim of the vessel were then prepared by applying adhesive primer in some versions. Adhesive was then administered and both parts were pressed together. Moving the two parts against each other in a circular motion leads to an even distribution of the adhesive.

### Synthesis of Mo-CORM

The CORM trisodiumtricarbonyl-[tris(isocyanoethylacetate)]molybdenum (Na₃Mo(CO)₃(CNCH₂CO₂)₃, Mo-CORM) was synthesized as previously published (Reiländer, ACS Biomaterials Science & Engineering, 2022). In brief, molybdenum hexacarbonyl was stirred in anhydrous acetonitrile to exchange three of its ligands for acetonitrile. These ligands were exchanged for EICA ligands. Purification and cation exchange yielded Mo-CORM as white solid.

### Sperm Preparation

Human sperm was collected from healthy volunteers in 100 ml specimen containers. Afterwards, the ejaculate was split into equal parts and filled into 15 mL gas tight falcon tubes, with one of the samples being exposed to one or more activated CORS. Then, both samples were analyzed for motility parameters via computer assisted sperm analysis (CASA), the static oxidation-reduction potential (sORP) via MiOXSYS and sperm DNA-fragmentation (SDF) via dispersion test with Halosperm G2 Kit after a predetermined time.

### Computer Assisted Sperm Analysis (CASA)

Total motility, progressive motility, VAP, and concentration of the samples were tested using a CEROS II (Hamilton Thorne, Beverly, MA) computer assisted sperm analysis (CASA) system. 6 μL of a sperm sample diluted 1:1 with Multipurpose Handling Medium-Complete (MHM-C, Fuji Film/Irvine Scientific, Santa Ana, CA) was investigated with the CASA system, and the level of mobility was assessed.

Static Oxidation-Reduction Potential (sORP) Analysis

An oxidative male infertility measuring System (MiOXSYS, Englewood, CO) was used to determine static oxidation-reduction potential (SORP) levels following manufacturer's specifications. 30 μL of sample were transferred to the sample application port onto a MiOXSYS sensor and analyzed for 2 minutes. The results were expressed in milli Volt (mV) and normalized to the seminal sperm concentration (mV/106 sperm/mL).

### DNA Fragmentation (SDF)

Sperm DNA fragmentation (SDF) was assessed via dispersion test using a Halosperm G2 Kit (From Halotech, Madrid, Spain) following manufacturer's specifications. In brief, the samples were diluted to a maximum of 20 mio/mL. The samples were, then, mixed with the prepared agarose. 8 μL of the mixture was transferred on slides and cooled down for 5 minutes. Different reagents were added and discarded after the set time: Denaturant agent was added for 7 minutes, lysis solution for 20 minutes, distilled water for 5 minutes, 70% ethanol for 2 minutes, 100% ethanol for 2 minutes, eosin staining solution for 7-10 minutes, and thiazine staining solution for 7-10 minutes. A minimum of 400 sperms were analyzed using an Olympus IX73 Inverted LED Fluorescence Microscope.

### Results

Here, the effects of CO released from a CO releasing system (CORS) on sperm viability and mobility parameters were investigated using an experimental design approach based on different amounts of CO gas (0.44-2.64 mL) and effective incubation time periods (60-270 minutes). Based on the fact that sperm cells require a certain balance of reactive oxygen species (ROS) for maintaining their motility, it was an important finding that exposure to CO did not impair sperm motility, progressive motility, and velocity during an extended processing time of up to 90 minutes at concentrations of 0.44-2.64 mL CO compared to untreated sperm. In the same process, the oxidation-reduction potential (sORP) and DNA fragmentation (SDF) were evaluated. According to WHO guidelines, liquefaction and processing should ideally take place within the first 60 minutes. However, this process may take longer than 60 minutes. Therefore, also longer time periods were considered when looking at CO treatment.

### 1. Spermatozoa Motility (Total Motility, Progressive Motility, and Average Path Velocity)

Sperm samples were exposed to three CORS or no CORS as a control (15 mg Mo-CORM; 1.32 mL CO) and tested at different exposure durations including 60-, 90-, 180-, and 270-min (FIGS. 6, 7, 9 and 10). Motility changed to 1.13±0.41-fold for 60 min, 0.99±0.03-fold for 90 min, 1.16±0.37-fold for 180 min, and 0.94±0.20-fold for 270 minutes. Progressive motility changed to 0.85±0.13-fold for 60 min, 0.92±0.17-fold for 90 min, 0.72±0.19-fold for 180 min, and 1.04±0.48-fold for 270 min. CASA was also used to determine the velocity of the sperm cells with a change to 0.94±0.09-fold for 60 min, 0.96±0.04-fold for 90 min, 0.79±0.13-fold for 180 min, and 0.97±0.08 for 270 min (FIG. 6A). To determine the effect of dose variations, we chose 90 min as exposure time for additional experiments. Here, sperm samples were exposed to either one, three, six, or no CORS as a control (5 mg, 15 mg, 30 mg, or 0 mg of Mo-CORM, corresponding to 0.44 mL, 1.32 mL, 2.64 mL, or 0 mL CO, respectively; FIGS. 6 and 8). Six was the upper limit of CORS the setup could hold at a time. Change in motility was to 0.98±0.12-fold for 0.44 mL and 0.90±0.12-fold for 2.64 mL, whereas change of progressive motility was to 0.97±0.12-fold for 0.44 mL and 0.88±0.10-fold for 2.64 mL. The change of VAP was to 1.01±0.08-fold for 0.44 mL and 1.03±0.03-fold for 2.64 mL (FIG. 6B).

FIG. 7 clearly confirms these observations made on the basis of FIG. 6.

### 2. Spermatozoa Viability Parameters (Oxidation-Reduction Potential and DNA Fragmentation)

During the same process oxidation-reduction potential and DNA-fragmentation were assessed. The impact of CO on the oxidation-reduction potential was shown in the change to 0.57±0.35-fold for 60 min, 0.91±0.12-fold for 90 min, 1.02±0.32-fold for 180 min, and 1.13±0.28-fold for 270 min. The DNA-fragmentation change was 0.77±0.07, 0.67±0.24-fold, 0.90±0.11-fold, and 0.90±0.19-fold for 60 min, 90 min, 180 min, and 270 min, respectively (FIG. 6C). For the dose variation samples we saw a change of oxidation-reduction potential to 0.89±0.17-fold for 0.44 mL and 0.59±0.24-fold for 2.64 mL. DNA-fragmentation changed to 0.83±0.09-fold for 0.44 mL and 1.31±0.55-fold for 2.64 mL (FIG. 6D). In summary, and in alignment with the WHO scheme it was observed that after 60 minutes there was a change in the oxidation-reduction potential to approximately 0.57±0.35-fold (1.32 mL). Additionally, DNA fragmentation exhibited a change to approximately 0.77±0.07-fold (1.32 mL). At a prolonged period of 90 min, a change in oxidation-reduction potential to 0.91±0.12-fold (1.32 mL) was observed. DNA fragmentation changed to 0.67±0.24-fold (1.32 mL).

In summary, the results demonstrate that the herein tested CO/Mo-CORM regimen (CO incubation duration and CO/Mo-CORM load) improve spermatozoan viability parameters. Namely, oxidation-reduction potential and spermatozoan DNA-fragmentation (SDF) was reduced at 60 min and 90 min CO incubation durations and 0.44 mL and 1.32 mL CO. This confirms the results depicted in FIGS. 2, 3, and 5. Thus, regarding spermatozoan viability parameters, 60 min or 90 min incubation durations with 0.44 mL or 1.32 mL CO may be preferred regimens.

Collectively, 60 min or 90 min incubation duration with 0.44 mL or 1.32 mL CO (corresponding to 5 mg or 15 mg Mo-CORM and 1 or 3 CORS, respectively) may be a preferred regimen, as they have no negative effects on spermatozoan motility, however, reduce spermatozoan DNA fragmentation and oxidation-reduction potential. Thus, the results demonstrated in FIGS. 6 and 7 clearly demonstrate that 60 min or 90 min incubation duration with 0.44 mL or 1.32 mL CO (corresponding to 5 mg or 15 mg Mo-CORM and 1 or 3 CORS, respectively) may be a preferred regimens as, they improve spermatozoan viability parameters without negatively affecting spermatozoan motility.

Further, an incubation duration of 60 min minutes fits perfectly into the daily laboratory routine of an IVF laboratory adhering to WHO guidelines (WHO laboratory manual for the examination and processing of human semen, World Health Organization, 2021). Thus, this treatment will likely improve success rates of artificial reproduction technology (ART) applications (such as in vitro fertilization; IVF) that are limited by high spermatozoan DNA fragmentation and oxidation-reduction potential.

Therefore, carbon monoxide (CO) when contacted to gametes like spermatozoa was surprisingly found by the inventors to lead to preservation of spermatozoa, especially, when spermatozoa were contacted with 0.44 mL or 1.32 mL CO for 60 min or 90 min. Consequently, spermatozoa that have been contacted with, inter alia, 0.44 mL or 1.32 mL CO for 60 min or 90 min have been shown to exhibit the desired characteristics (compared with spermatozoa that were not contacted with CO) and are thus particularly useful for ART (applications).

3. Effect of CO-Treatment on the Viability of Spermatozoa from Subjects with Reduced Spermatozoan Viability

Panels D and E of FIGS. 7 and 8 depict the oxidation-reduction potential and DNA fragmentation measurements of untreated/Co-treated sperm samples from individual subjects. Here, high variation between untreated sperm samples of different individuals can be observed for both oxidation-reduction potential and DNA fragmentation, clearly demonstrating that among the tested subjects the sperm of several subjects suffers from extraordinarily high oxidative stress when untreated. Here, particularly untreated sperm samples 42_1 and 38_7 show drastically elevated oxidation-reduction potential and DNA fragmentation levels, as compared to the majority of samples. It is conceivable that these samples are derived from subjects suffering from infertility/below average fertility. CO treatment caused a strong reduction of both oxidation-reduction potential and DNA fragmentation values for samples 42_1 and 38_7. This clearly demonstrates that CO treatment is able to aid subjects with sperm having severely compromised viability parameters. As such, it is conceivable that CO treatment may be used to treat/prevent (gamete) diseases that are characterized by elevated oxidation-reduction potential and/or DNA fragmentation in gametes. This includes, inter alia, treating or reducing male infertility. Further, it is conceivable that CO treatment of gametes may prevent congenital abnormalities and/or aneuploidy in any progeny deriving from said gametes that were treated with CO.

FIGS. 9 and 10 further confirm the effectiveness of CO treatment on both oxidation-reduction potential and DNA fragmentation, respectively. These figures depict correlative analyses of the effect of the CO treatment (difference of treated and untreated samples; y-axis) with the respective measurements of the untreated sperm samples (x-axis). Both figures focus on 60 min and 90 min incubation durations, as these regimens were showing pronounced effectiveness of CO-treatment before (FIG. 7). FIGS. 9 and 10 demonstrate that CO treatment is particularly effective in subjects that suffer from high sperm oxidation-reduction potential and/or DNA-fragmentation.

1. Method for the preservation of gametes, the method comprising the steps of
a) providing/obtaining gametes or a sample comprising said gametes in a container and
b) contacting said gametes or said sample comprising said gametes in said container with carbon monoxide.

2. The method according to claim 1, wherein said gametes or said sample comprising said gametes comprise spermatozoa or egg cells, preferably wherein said spermatozoa are comprised within seminal fluid, ejaculate and/or a buffer/buffer system.

3. The method according to claims 1 and 2, wherein said carbon monoxide is released from a carbon monoxide releasing molecule and/or wherein said carbon monoxide is directly supplied to said container via a separate tube.

4. The method according to any one of claims 1 to 3, wherein said carbon monoxide forms a gas layer over said gametes or over said sample comprising said gametes.

5. The method according to claims 3 and 4, wherein said carbon monoxide releasing molecule is preferably a metal carbonyl compound, even more preferably a molybdenum carbonyl compound, most preferably Na₃Mo(CO)₃(CNCH₂CO₂H)₃.

6. The method according to any one of claims 3 to 5, wherein said carbon monoxide releasing molecule is to be contacted with FeCl₃, Ce(SO₄)₂ or H₂O₂, preferably with FeCl₃ thereby releasing said carbon monoxide.

7. The method according to any one of claims 1 to 6, wherein said gametes or said sample comprising said gametes are contacted with said carbon monoxide for about 15 to about 120 minutes, preferably for about 30 to about 90 minutes, more preferably for about 30 to about 60 minutes.

8. The method according to any one of claims 1 to 7, wherein said gametes or said sample comprising said gametes are contacted with said carbon monoxide at about 25 °C to about 35 °C.

9. The method according to any one of claims 1 to 8, wherein the amount of carbon monoxide applied to said gametes or said sample comprising said gametes is between about 20 μmol and about 500 μmol, about 40 μmol and about 400 μmol, preferably between about 45 μmol and about 270 μmol, preferably between about 60 μmol and about 180 μmol.

10. The method according to any one of claims 1 to 9, wherein said gametes are human gametes.

11. The method according to any one of claims 1 to 9, wherein said gametes are gametes from cattle, horses, pigs, sheep, goats, camels, alpacas, dogs, cats, birds, or fish.

12. The method according to any one of claims 1 to 11, wherein the preservation of said gametes is assessed via determination of motility, DNA-fragmentation, spermiogram and/or oxidation-reduction potential of said gametes.

13. A method for reducing DNA-fragmentation and/or oxidation-reduction potential of gametes in an artificial reproductive technology, wherein said gametes are or a sample comprising said gametes is contacted with carbon monoxide.

14. Use of carbon monoxide for reducing DNA-fragmentation and/or oxidation-reduction potential of gametes in an assisted reproductive technology, wherein said gametes are or a sample comprising said gametes is contacted with carbon monoxide.

15. The method according to claim 13, or the use according to claim 14, wherein said assisted reproductive technology comprises in vitro fertilization, intracytoplasmic sperm injection, intrauterine insemination, frozen embryo replacement, preimplantation genetic testing, in vitro maturation of oocytes, frozen oocyte replacement, gamete intrafallopian transfer, zygote intrafallopian transfer, and/or cryopreservation.

16. The method according to claim 13 or 15, or the use according to claim 14 or 15, wherein reducing DNA-fragmentation and/or oxidation-reduction potential of gametes maintains/increases motility of said gametes.

17. The method according to any one of claims 13, 15, and 16, or the use according to any one of claims 14 to 16, wherein reducing DNA-fragmentation and/or oxidation-reduction potential of gametes reduces the risk of congenital abnormality and/or aneuploidy in an artificial reproductive technology.

18. Carbon monoxide for use in preventing congenital abnormality and/or aneuploidy.

19. Carbon monoxide for use in treating/reducing male infertility.

20. Carbon monoxide for use in treating and/or preventing a disease caused by/linked to elevated DNA fragmentation and/or oxidation-reduction potential of gametes.

21. Carbon monoxide for use in treating and/or preventing a disease of gametes caused by/linked to elevated ROS levels.

22. The carbon monoxide for use according to claim 20 or 21, wherein said disease is caused by/linked to elevated stress levels or wherein the individual to be treated suffers from stress.

23. The carbon monoxide for use according to claim 22, wherein said disease is caused by/linked to elevated psychological and/or physiological stress levels or wherein the individual to be treated suffers from psychological and/or physiological stress.

24. The carbon monoxide for use according to any one of claims 18 to 23, wherein preventing congenital abnormality and/or aneuploidy, treating/reducing male infertility, or treating/preventing said disease is characterized by contacting gametes with carbon monoxide.

25. The carbon monoxide for use according to claim 24, wherein said gametes are contacted with carbon monoxide in vitro/ex corpore.

26. The method according to any one of claims 13, and 15 to 17, the use according to any one of claims 14 to 17, or the carbon monoxide for use of claim 25, wherein said gametes are or said sample comprising said gametes is contacted with said carbon monoxide less than 10 min after collection of said gametes in said container, preferably less than 5 min, more preferably less than 3 min, more preferably less than 2 min, more preferably less than 1 min.

27. The carbon monoxide for use according to claim 24, wherein said gametes are or said sample comprising said gametes is contacted with carbon monoxide in vivo.

28. The carbon monoxide for use according to claim 27, wherein said carbon monoxide is administered systemically or transdermally to said gametes.
